(19) United States
(12) Patent Application Publication
(10) Pub. No.: US 2023/0293010 A1
(43) Pub. Date: Sep. 21, 2023

(54) NON-FACE-TO-FACE BODY TEMPERATURE CHANGE MANAGEMENT PLATFORM SERVICE SYSTEM AND METHOD

(71) Applicant: AMOSENSE CO.,LTD, Cheonan-si (KR)

(72) Inventor: Jin Sung YANG, Cheonan-si (KR)

(21) Appl. No.: 17/999,695

(22) PCT Filed: May 28, 2021
(86) PCT No.: PCT/KR2021/006649

§ 371 Date: Dec. 30, 2022

(30) Foreign Application Priority Data
Jun. 4, 2020 (KR) 10-2020-0067552
Jun. 4, 2020 (KR) 10-2020-0067553
Jun. 12, 2020 (KR) 10-2020-0071295
Jun. 12, 2020 (KR) 10-2020-0071297

(51) Int. Cl. A61B 5/00; A61B 5/01; A61B 5/0205

(52) U.S. Cl. CPC A61B 5/0008; A61B 5/01; A61B 5/0022; A61B 5/02055

(57) ABSTRACT

A non-face-to-face body temperature change management platform service system and method are provided. The non-face-to-face body temperature change management platform service system according to one embodiment of the present invention comprises: an electronic device that is located around a user and collects and transmit body temperature information of the user and location information thereof; and a data server that receives the body temperature information and the location information from the electronic device and monitors an amount of change (Δ) in body temperature compared to the average body temperature of the user.

### TECHNICAL FIELD

The present invention relates to a non-face-to-face body temperature change management platform service system and method.

### BACKGROUND ART

Recently, viral infections have been spreading all over the world. Since one of the early symptoms of such an infection is fever, continuous management and supervision of changes in body temperature are necessary to check for the infection or manage confirmed or suspected people.

Further, such an infection has an incubation period of about two weeks, and thus it is necessary to monitor changes in body temperature for a certain period of time even when no special symptoms appear. Therefore, a method of automatically transmitting temperature information for monitoring is necessary.

### PRIOR ART DOCUMENTS

KR 1978264 B1 (registered on May 8, 2019)

### DISCLOSURE

### Technical Problem

The present invention is directed to providing a non-face-to-face body temperature change management platform service system and method for monitoring changes in the body temperature and location of a user in real time.

### Technical Solution

One aspect of the present invention provides a non-face-to-face body temperature change management platform service system including an electronic device that is located near a user and configured to collect and transmit body temperature information of the user and location information thereof and a data server configured to receive the body temperature information and the location information from the electronic device and monitor a body temperature variation (Δ) compared to an average body temperature of the user.

The data server may monitor and classify the body temperature variation (Δ) as a case of a first threshold value or more, a case of a second threshold value or more and less than the first threshold value, or a case of less than the second threshold value, and when the body temperature variation (Δ) of the user is the case of the first threshold value or more, may determine the user to be a suspected person and alarm an integrated control server and a manager terminal with a corresponding fact.

An application dedicated to body temperature change and location monitoring may be installed on the manager terminal, and the manager terminal may receive results of monitoring the body temperature variation (Δ) from the data server through the dedicated application.

The integrated control server may include a server of a center for disease control and prevention and a disinfection manager monitoring server.

The non-face-to-face body temperature change management platform service system may further include a patch-type thermometer that is attached to the user's body to measure and transmit the body temperature, and the electronic device may be a user terminal that is tapped on the patch-type thermometer to form a magnetic field, requests body temperature measurement from the patch-type thermometer to receive the measured temperature information, and collects Global Positioning System (GPS) location information through a GPS receiver unit to transmit the body temperature information and the location information.

The patch-type thermometer may be driven in an energy harvesting manner based on inductive coupling with the magnetic field.

The patch-type thermometer may further include a detection sensor configured to measure bio-information of the user, and the detection sensor may measure at least one of the user's heart rate, electrocardiogram, oxygen saturation, and blood pressure.

The non-face-to-face body temperature change management platform service system may further include a patch-type thermometer that is attached to the user's body to periodically measure and transmit the body temperature, and the electronic device may be a beacon gateway that is installed in a predetermined place to provide corresponding location information, receives the body temperature information transmitted by the patch-type thermometer, and transmits the body temperature information and the installation location information together.

The non-face-to-face body temperature change management platform service system may further include a user terminal configured to receive the body temperature information from the patch-type thermometer, collect GPS location information through a GPS receiver unit, and transmit the body temperature information and the GPS location information to the data server.

The patch-type thermometer may measure the body temperature at certain time intervals and perform advertising transmission of the body temperature at certain periods.

The beacon gateway may provide fixed location information for each installation place in each indoor floor and each indoor space.

The electronic device may include a battery configured to supply power and a wireless charger unit configured to charge the battery.

The non-face-to-face body temperature change management platform service system may further include a thermometer configured to periodically measure and transmit the body temperature of the user, and the electronic device may be a location tracker that is worn by the user, collects the body temperature information measured by the thermometer, collects GPS location information through a GPS receiver unit, and transmits the body temperature information and the location information.

The data server may receive the body temperature information and the location information of the user from the location tracker through a Sigfox Internet of Things (IoT) server.

The non-face-to-face body temperature change management platform service system may further include a beacon gateway that is installed in a predetermined place to provide corresponding location information, receives the body temperature information transmitted by the thermometer, and transmits the body temperature information and the installation location information together.

The thermometer may be any one of a patch-type thermometer attached to the user's body and a band-type thermometer worn on the user's wrist.

The electronic device may be a band-type smart thermometer that is worn by the user to periodically measure the body temperature, collects GPS location information through a GPS receiver unit, and transmits the measured body temperature information and the location information. The smart thermometer may transmit the body temperature information and the location information of the user to a Sigfox IoT server, and the data server may receive the body temperature information and the location information of the user from the Sigfox IoT server.

Another aspect of the present invention provides a non-face-to-face body temperature change management platform service method including collecting, by an electronic device near a user, body temperature information of the user and location information of the electronic device and transmitting the body temperature information and the location information to a data server, and receiving, by the data server, the body temperature information and the location information from the electronic device and monitoring a body temperature variation (Δ) compared to an average body temperature of the user.

### Advantageous Effects

According to the present invention, body temperature information measured by a thermometer is periodically transmitted so that a change in the body temperature of a user can be managed in real time. Accordingly, it is possible to prevent infectious diseases showing the symptom of abnormal body temperature.

According to the present invention, it is possible to find a travel route of a confirmed person or a suspected person who is expected to be confirmed, by managing location information as well as body temperature information. Accordingly, it is possible to prevent the proliferation of infectious diseases at an early stage.

According to the present invention, a body temperature of a user is monitored using only changes in the body temperature with respect to an average body temperature of the user, and thus the body temperature information which is one kind of bio-information of the user is not stored in a server. Accordingly, it is possible to improve the reliability of personal information security.

According to the present invention, the amount of change in the body temperature of a user is monitored to detect an intentional regulation of the body temperature made by taking a fever reducer through the trend of repeated rises and falls in the body temperature. Accordingly, it is possible to improve efficiency and reliability of disease control.

According to the present invention, measured body temperature information is transmitted to a data server through a user terminal carried by a user so that the body temperature information can be transmitted without any additional device as long as a dedicated application is installed on the user terminal. Accordingly, changes in the body temperature can be effectively managed.

According to the present invention, a smart thermometer or a location tracker transmits body temperature information on the basis of a low power wide area (LPWA). Accordingly, power consumption of the smart thermometer or the location tracker can be minimized, and the smart thermometer or the location tracker can be used for a long time. Therefore, it is possible to eliminate the hassle of frequent battery charging, replacement, etc. and reduce maintenance costs.

According to the present invention, body temperature information is measured by a smart thermometer worn by a user and is directly transmitted to a data server without a specific manipulation or any additional device. Accordingly, it is possible to improve convenience of body temperature measurement and body temperature change management.

According to the present invention, measured body temperature information is transmitted to a data server through a pre-installed beacon gateway. Accordingly, body temperature information can be automatically transmitted without a specific manipulation or together with specific indoor location information. Therefore, changes in body temperature can be effectively monitored.

According to the present invention, measured body temperature information is transmitted to a data server through a location tracker worn by a user, and thus the body temperature information can be transmitted to a user who does not have a personal portable terminal or is blocked. Accordingly, changes in body temperature can be effectively managed.

According to the present invention, bio-signals of a heart rate, an electrocardiogram, an oxygen saturation, etc. are measured and managed together with body temperature information so that infectious diseases and other diseases can be rapidly checked in real time. Accordingly, it is possible to efficiently manage a user's health.

According to the present invention, a wireless charging function is provided in a beacon gateway or a location tracker so that the beacon gateway or the location tracker can be repeatedly used after a patch-type thermometer is replaced. Accordingly, maintenance costs can be minimized, and it is possible to improve economic benefits and manageability.

According to the present invention, a wireless charging function is provided in a smart thermometer. Accordingly, it is possible to omit an annoying preparation operation for charging the smart thermometer, and convenience of a user can be improved.

### DESCRIPTION OF DRAWINGS

FIG. 1 is a diagram schematically showing a configuration of a non-face-to-face body temperature change management platform service system according to a first embodiment of the present invention.

FIG. 2 is a block diagram showing a detailed configuration of a patch-type thermometer of FIG. 1.

FIG. 3 is a block diagram showing a detailed configuration of a user terminal of FIG. 1.

FIG. 4 is a block diagram showing a detailed configuration of a service server of FIG. 1.

FIG. 5 is a diagram illustrating a body temperature measurement process using the patch-type thermometer and the user terminal of FIG. 1.

FIG. 6 is a view showing a first example of a manager web screen of a data server of FIGS. 1, 11, 17, and 23.

FIG. 7 is a view showing a second example of a manager web screen of the data server of FIGS. 1, 11, 17, and 23.

FIG. 8 is a view showing a third example of a manager web screen of the data server of FIGS. 1, 11, 17, and 23.

FIG. 9 is a view showing a fourth example of a manager web screen of the data server of FIGS. 1, 11, 17, and 23.

FIG. 10 is a flowchart illustrating a non-face-to-face body temperature change management platform service method according to the first embodiment of the present invention.

FIG. 11 is a diagram schematically illustrating a non-face-to-face body temperature change management platform service system according to a second embodiment of the present invention.

FIG. 12 is a block diagram showing a detailed configuration of a patch-type thermometer of FIG. 11.

FIG. 13 is a block diagram showing a detailed configuration of a beacon gateway of FIG. 11.

FIG. 14 is a block diagram showing a detailed configuration of a user terminal of FIG. 11.

FIG. 15 is a block diagram showing a detailed configuration of the data server of FIG. 11.

FIG. 16 is a flowchart illustrating a non-face-to-face body temperature change management platform service method according to the second embodiment of the present invention.

FIG. 17 is a diagram schematically illustrating a non-face-to-face body temperature change management platform service system according to a third embodiment of the present invention.

FIG. 18 is a block diagram showing a detailed configuration of a thermometer of FIG. 17.

FIG. 19 is a block diagram showing a detailed configuration of a location tracker of FIG. 17.

FIG. 20 is a block diagram showing a detailed configuration of a beacon gateway of FIG. 17.

FIG. 21 is a block diagram showing a detailed configuration of the data server of FIG. 17.

FIG. 22 is a flowchart illustrating a non-face-to-face body temperature change management platform service method according to the third embodiment of the present invention.

FIG. 23 is a diagram schematically illustrating a non-face-to-face body temperature change management platform service system according to a fourth embodiment of the present invention.

FIG. 24 is a block diagram showing a detailed configuration of a smart thermometer of FIG. 23.

FIG. 25 is a block diagram showing a detailed configuration of a beacon gateway of FIG. 23.

FIG. 26 is a block diagram showing a detailed configuration of a data server of FIG. 23.

FIG. 27 is a flowchart illustrating a non-face-to-face body temperature change management platform service method according to the fourth embodiment of the present invention.

### MODES OF THE INVENTION

Hereinafter, embodiments of the present invention will be described in detail with reference to the accompanying drawings so that those of ordinary skill in the art can readily implement the present invention. The present invention can be implemented in a variety of different forms and is not limited to the embodiments set forth herein. In the drawings, parts unrelated to the description are omitted for clear description. Throughout the specification, like reference numerals refer to like components.

A non-face-to-face body temperature change management platform service system according to embodiments of the present invention is a platform service system for periodically transmitting body temperature information measured through a patch-type thermometer for the purpose of management and may automatically transmit body temperature information measured from a user to a data server through an electronic device so that changes in the body temperature of the user can be managed. Here, the change in the body temperature is a body temperature variation Δ compared to an average body temperature of the user.

The electronic device is a device that is near the user and collects and transmits the body temperature information of the user and location information thereof. The electronic device may be at least one of a user terminal, a beacon gateway, a location tracker, and a band-type smart thermometer. Also, the data server may receive the body temperature information and the location information from the electronic device and monitor the body temperature variation Δ compared to the average body temperature of the user.

In this way, the non-face-to-face body temperature change management platform service system according to embodiments of the present invention can effectively manage changes in the body temperature of the user in real time. Accordingly, it is possible to estimate infectious diseases which show the symptom of abnormal body temperature, and the infectious diseases can be prevented.

According to first to third embodiments, patch-type thermometers 100A, 100B, and 100C₂ may be implemented in the form of a patch and attached to the user's body. To this end, the patch-type thermometers 100A, 100B, and 100C₂ may have adhesive strength or viscosity, or bodies thereof may be made of medical silicone and the like and then attached to the user's body using a supplementary adhesive member.

Also, a negative temperature coefficient (NTC) thermistor used in the patch-type thermometers 100A and 100B and a thermometer 100C according to the first to third embodiments may have a deviation in the absolute value compared to a contactless thermometer employing infrared rays, laser, etc. However, the NTC thermistor can accurately measure a temperature variation and thus is appropriate for measuring the body temperature variation Δ of the user.

For example, according to the first to fourth embodiment, inaccurate information on a body temperature may be measured by the patch-type thermometer 100A and 100B, the thermometer 100C, and a smart thermometer 100D unlike general contactless thermometers. Data servers 300A, 400B, 400C, and 300D may infer health status of the user through the trend of changes in the body temperature variation Δ and the like input from the patch-type thermometer 100A and 100B, the thermometer 100C, and the smart thermometer 100D. In other words, even when a measured temperature is different from an actually measured body temperature, it is possible to monitor the body temperature variation Δ. Accordingly, it is possible to see that the body temperature drastically rises to a certain level or above or repeatedly rises and falls due to internal use of a fever reducer.

Meanwhile, a non-face-to-face body temperature change management platform service method according to embodiments of the present invention is performed in the above-described system and includes a first step in which an electronic device near a user collects and transmits body temperature information of the user and location information thereof to a data server and a second step in which the data server receives the body temperature information and the location information and monitors a body temperature variation Δ compared to an average body temperature of the user.

In particular, the non-face-to-face body temperature change management platform service method according to embodiments of the present invention may be implemented as a software (S/W) program for performing each of the steps. In this case, such programs may be stored in a computer-readable recording medium or transmitted as a computer data signal combined with a carrier wave in a transmission medium or a communication network. The computer-readable recording medium includes any type of recording device in which data readable by a computer system is stored, for example, a read only memory (ROM), a random access memory (RAM), a compact disc (CD)-ROM, a digital versatile disc (DVD)-ROM, a DVD-RAM, a magnetic tape, a floppy disk, a hard disk, an optical data storage device, etc.

However, further details of the non-face-to-face body temperature change management platform service system and method according to embodiments of the present invention will be described according to the first to fourth embodiments. When the same description is included in each embodiment, the description in the later embodiment may be replaced with the description in the previous embodiment.

### First Embodiment

A non-face-to-face body temperature change management platform service system 10A according to the first embodiment of the present invention is a platform service system for periodically transmitting body temperature information measured through the patch-type thermometer 100A for the purpose of management and may automatically transmit body temperature information measured from a user to a data server 300A through a user terminal 200A so that changes in the body temperature of the user can be managed.

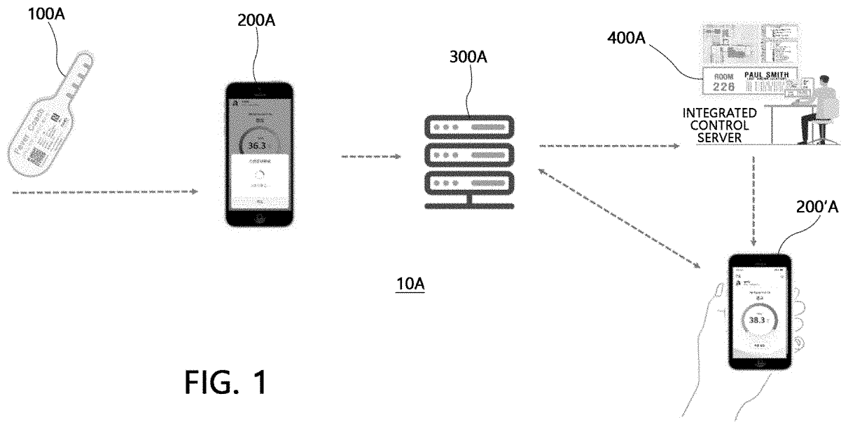

To this end, the non-face-to-face body temperature change management platform service system 10A according to the first embodiment of the present invention includes the patch-type thermometer 100A, the user terminal 200A, and the data server 300A as shown in FIG. 1.

The patch-type thermometer 100A may be attached to the user's body and measure the body temperature. Here, the patch-type thermometer 100A may be driven in an energy harvesting manner based on inductive coupling with a magnetic field formed from the user terminal 200A. In other words, the patch-type thermometer 100A may be driven by the user terminal 200A on the basis of near field communication (NFC).

Accordingly, the patch-type thermometer 100A does not require a power supply for operating, and thus the overall weight can be reduced. Also, since a battery is omitted, the patch-type thermometer 100A may be implemented in an ultra-thin form.

After starting operation, the patch-type thermometer 100A may measure the body temperature according to a request of the user terminal 200A and transmit the body temperature to the user terminal 200A. For example, the patch-type thermometer 100A may transmit body temperature information that is obtained through self-measurement by tapping the user terminal 200A on the patch-type thermometer 100A, to the user terminal 200A on the basis of NFC.

When the user terminal 200A is tapped on the patch-type thermometer 100A, the user terminal 200A may form a magnetic field and request body temperature measurement from the patch-type thermometer 100A. For example, the user terminal 200A may be a portable electronic device such as a smartphone. In this case, the user terminal 200A may communicate with the patch-type thermometer 100A on the basis of NFC. Also, the user terminal 200A may receive body temperature information transmitted by the patch-type thermometer 100A and transmit the body temperature information to the data server 300A.

Also, the user terminal 200A may collect Global Positioning System (GPS) location information from GPS satellites (not shown) and transmit the GPS location information to the data server 300A. Here, the user terminal 200A may include a GPS receiver unit. As an example, the user terminal 200A may transmit location information at shorter periods than body temperature information. As another example, the user terminal 200A may collect location information at shorter periods than body temperature information and simultaneously transmit the location information together with the body temperature information.

In this way, the non-face-to-face body temperature change management platform service system 10A according to the first embodiment of the present invention can find, using location information, a travel route of a person subject to self-quarantine at an immigration stage, a person confirmed with an infectious disease, such as COVID19, or a suspected person who is expected to be confirmed. Accordingly, it is possible to easily specify people having a travel route overlapping that of the confirmed person or the suspected person and prevent the proliferation of infectious diseases at an early stage (hereinafter, a “first advantage”).

Also, an application dedicated to body temperature change and location monitoring may be installed on the user terminal 200A. Here, the user terminal 200A may receive monitoring results regarding the body temperature variation Δ from the data server 300A through the dedicated application. For example, the user terminal 200A may receive a measured body temperature, the trend of changes in the body temperature, a state suspected of a fever, etc. from the data server 300A.

In this way, the non-face-to-face body temperature change management platform service system 10A according to the first embodiment of the present invention can transmit body temperature information without an additional device for collecting and transmitting the body temperature as long as the dedicated application is installed on the user terminal 200A. Accordingly, changes in the body temperature can be effectively managed (hereinafter, a “second advantage”).

FIG. 1 shows that the user terminal 200A transmits body temperature information and a manager terminal 200′A receives monitoring results from the data server 300A through the dedicated application.

The data server 300A may receive the body temperature information of the user and the location information from the user terminal 200A for the purpose of management. The data server 300A may monitor the body temperature variation Δ of the user. Here, the data server 300A may track the location of the user and find a travel route of the user in accordance with the location information.

Also, the data server 300A may monitor and classify the body temperature variation Δ as a case of a first threshold value or more, a case of a second threshold value or more and less than the first threshold value, or a case of less than the second threshold value. For example, the data server 300A may classify the body temperature variation Δ as a case of 1.5° C. or higher, a case between 1° C. and 1.5° C., and a case of lower than 1° C.

In this way, the non-face-to-face body temperature change management platform service system 10A according to the first embodiment of the present invention stores and uses a body temperature variation compared to an average body temperature that varies depending on each individual. Since it is not possible to identify a body temperature which is bio-information of a corresponding user from the body temperature variation alone, the reliability of personal information security can be improved.

When the body temperature variation Δ of the user is the case of the first threshold value or more, the data server 300A may determine the user to be a suspected person. Here, the first threshold value may be a body temperature value from which a change in the body temperature may be determined to be a fever. As an example, the first threshold value may be set to a value that differs by 2° C. or more from a normal body temperature. In other words, when the user is suspected to have a fever in accordance with a change in the body temperature, the data server 300A may determine the user to be a suspected person. In this case, the data server 300A may alarms an integrated control server 400A, such as control headquarters installed in a center for disease control and prevention, companies, hospitals, etc., the user terminal 200A, and the manager terminal 200′A with the corresponding fact.

Also, when the body temperature variation Δ is the case of greater than or equal to the second threshold value and less than the first threshold value, the data server 300A may determine the user to be a target of interest. Here, the second threshold value may be a smaller value than the first threshold value. In other words, when the user does not have a fever but his or her body temperature variation is relatively large, the data server 300A may determine the user to be a target of interest. In this case the data server 300A may alarm the integrated control server 400A, the user terminal 200A, and the manager terminal 200′A with the corresponding fact.

Also, when the body temperature variation Δ is the case of less than the second threshold value, the data server 300A may determine the user to be a normal user. Here, the second threshold value may be a body temperature value corresponding to a usual body temperature change of each individual. For example, the second threshold value may be a temporary body temperature change caused by fast walking, exercise, etc.

In this way, the non-face-to-face body temperature change management platform service system 10A according to the first embodiment of the present invention can detect an intentional regulation of the body temperature made by taking a fever reducer through the trend of repeated rises and falls in the body temperature. Accordingly, it is possible to improve efficiency and reliability of disease control.

Meanwhile, the non-face-to-face body temperature change management platform service system 10A may further include the integrated control server 400A.

The integrated control server 400A may receive a state of the user from the data server 200A. For example, the integrated control server 400A may include a server of the center for disease control and prevention and a disinfection manager monitoring server. The integrated control server 400A may receive body temperature information and location information of the user together.

Also, the integrated control server 400A may transmit a precise examination request, an isolation request, etc. to the user terminal or the manager terminal 200′A through an emergency message or a phone call. In this case, the application dedicated to body temperature change and location monitoring may be installed on the user terminal or the manager terminal 200′A, and the user terminal or the manager terminal 200′A may receive monitoring results regarding the body temperature variation Δ from the server 300A through the dedicated application. Here, the manager terminal 200′A is a portable electronic device, such as a personal computer or a smartphone, and may be an electronic device used or owned by a manager

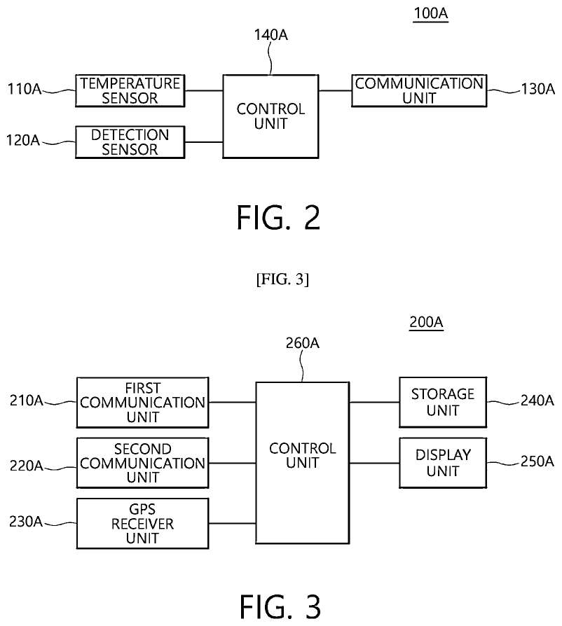

Referring to FIG. 2, the patch-type thermometer 100A is manufactured on the basis of a flexible circuit board and may include a temperature sensor 110A, a communication unit 130A, and a control unit 140A.

The temperature sensor 110A may come into contact with a user's body to measure a temperature. Here, the temperature sensor 110A may be a digital temperature sensor. For example, the temperature sensor 110A may be a negative temperature coefficient (NTC) thermistor.

The communication unit 130A may perform NFC with the user terminal 200A. Here, the communication unit 130A may be an NFC antenna. For example, the communication unit 130A may be an antenna pattern provided on the flexible circuit board. The communication unit 130A may not only transmit the body temperature information measured by the temperature sensor 100A but also generate driving power required for the patch-type thermometer 100A.

More specifically, the communication unit 130A may be inductively coupled with a magnetic field formed from the terminal and generate power in an energy harvesting manner based on the inductive coupling. In other words, when the user terminal 200A is tapped on the communication unit 130A, the communication unit 130A may be inductively coupled with the magnetic field from the user terminal 200A to generate power for driving the patch-type thermometer 100A.

Also, the communicator 130A may transmit the body temperature information measured by the temperature sensor 110A to the user terminal 200A on the basis of NFC.

The control unit 140A may control the above-described overall operations of the patch-type thermometer 100A in communication with the temperature sensor 110A and the communication unit 130A. For example, the control unit 140A may be a driving chip of the patch-type thermometer 100A.

Meanwhile, the patch-type thermometer 100A may further include a detection sensor 120A for measuring bio-information of the user. Here, the bio-information includes at least one of a heart rate, an electrocardiogram, an oxygen saturation, and a blood pressure but is not limited thereto.

The patch-type thermometer 100A may transmit the measured bio-information to the user terminal 200A together with the body temperature information.

In this way, the non-face-to-face body temperature change management platform service system 10A according to the first embodiment of the present invention allows a rapid check of convalescence from general diseases as well as infectious diseases in real time. Accordingly, healthcare of a user can be improved (hereinafter, a “third advantage”).

Referring to FIG. 3, the user terminal 200A may include a first communication unit 210A, a second communication unit 220A, a GPS receiver unit 230A, a storage unit 240A, a display unit 250A, and a control unit 260A.

The first communication unit 210A may wirelessly communicate with the patch-type thermometer 100A using NFC. While communicating with the patch-type thermometer 100A, the first communication unit 210A may form a magnetic field toward the patch-type thermometer 100A. In other words, the first communication unit 210A may provide power to the patch-type thermometer 100A due to the formed magnetic field.

The second communication unit 220A may perform long-range wireless communication with the user terminal 200A. For example, a communication unit 310A may communicate with the user terminal 200A through a public communication network using a communication protocol such as WiFi, Long Term Evolution (LTE), fifth generation (5G), etc.

The GPS receiver unit 230A may receive location information from the GPS satellites. The GPS receiver unit 230A may store the received location information in the storage unit 240A. The GPS receiver unit 230A may store time-specific location information to transmit later the location information to the data server 300A at once.

The storage unit 240A may store the body temperature information and the location information transmitted by the patch-type thermometer 100A. Here, the storage unit 240A may store the body temperature information and the location information during a preset period. In other words, the storage unit 240A may store the body temperature information and the location information for a certain time even after the body temperature information and the location information is transmitted to the data server 300A.

The display unit 250A may display monitoring results provided by the data server 300A. For example, the display unit 250A may be a display panel.

The control unit 260A may control the above-described overall operations of the user terminal 200A in communication with the first communication unit 210A, the second communication unit 220A, the GPS receiver unit 230A, the storage unit 240A, and the display unit 250A.

Here, the control unit 260A may be a dedicated application installed on the user terminal 200A. In other words, the control unit 260A may be S/W that is run by a processor of the user terminal 200A.

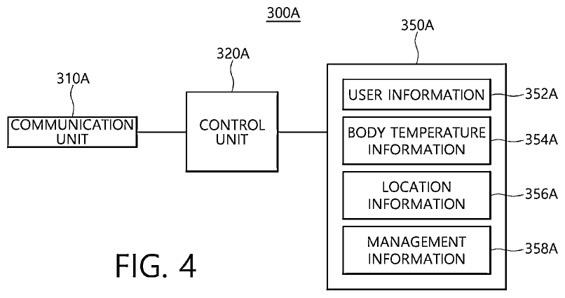

Referring to FIG. 4, the data server 300A may include the communication unit 310A, a control unit 320A, and a database 350A.

The communication unit 310A may perform long-range wireless communication with the user terminal 200A. For example, the communication unit 310A may communicate with the user terminal 200A through a public communication network.

The control unit 320A may control the above-described overall operations of the data server 300A in communication with the communication unit 310A and the database 350A. For example, the control unit 320A may be an artificial intelligence (AI) module dedicated to body temperature management.

The database 350A may include user information 352A, body temperature information 354A, location information 356A, and management information 358A.

The user information 352A may be information of the user to whom the patch-type thermometer 100A is attached. For example, the user information 352A may include the user's name, age, sex, hospitalization information, and immigration information. Here, the hospitalization information may include a date and time of hospitalization, a reason for hospitalization, and the name of a diagnosed disease. Also, the immigration information may include a date and time of entry and exit, a country of entry and exit, and a period of sojourn.

The body temperature information 354A may be body temperature information measured by the patch-type thermometer 100A and transmitted through the user terminal 200A. Here, the body temperature information may include the trend of period-specific body temperature variations A.

The location information 356A may be GPS location information received from the user terminal 200A. The location information 356A may include time-specific location information.

The management information 358A may be disease control- or disinfection-related institution information. For example, the management information 358A may include a server of the center for disease control and a server of the disinfection manager monitoring, or communication availability information (eg, phone number, etc.) of a corresponding institution or person in charge. In addition, the management information 358A may include a server of the regional management institution or communication availability information (eg, phone number, etc.) of a corresponding institution or person in charge.

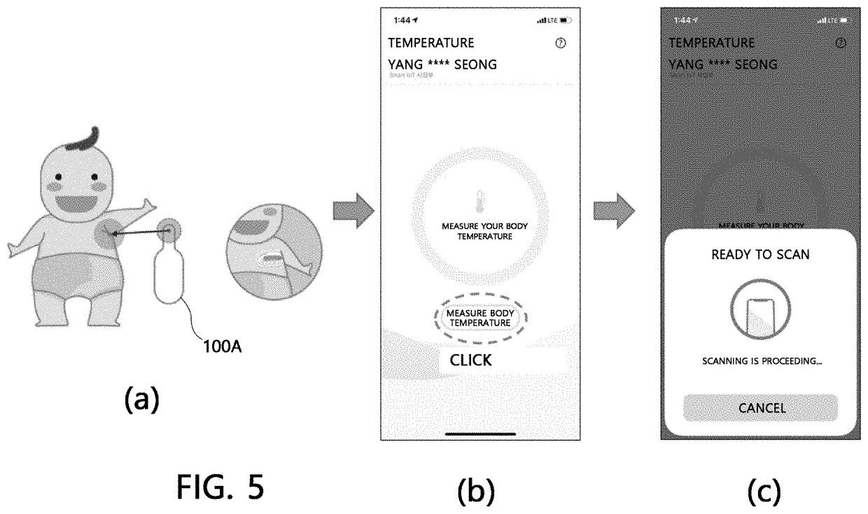

A body temperature measurement process using the patch-type thermometer 100A and the user terminal 300A will be described with reference to FIG. 5. First, the patch-type thermometer 100A may be attached to a user's body. For example, the patch-type thermometer 100A may be attached to the user's armpit (see FIG. 5A).

When the user terminal 200A is tapped on the patch-type thermometer 100A, the patch-type thermometer 100A starts operating in an energy harvesting manner At the same time, the dedicated application is run on the user terminal 200A so that a selection button for body temperature measurement may be displayed through the dedicate application (see FIG. 5B).

When the user or the manager clicks on the selection button, the user terminal 200A may request body temperature measurement from the patch-type thermometer 100A. Here, the user terminal 200A may receive body temperature information measured by the patch-type thermometer 100A (see FIG. 5C).

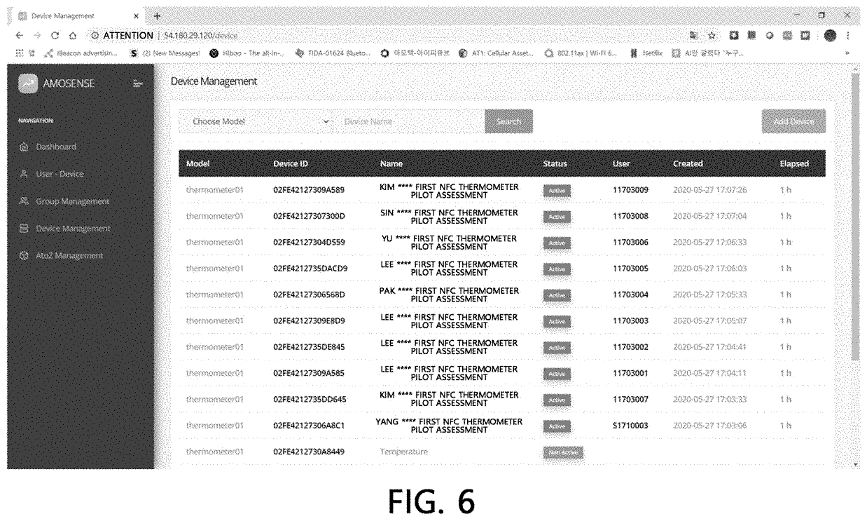

Referring to FIGS. 6 to 9, the data server 300A may readily manage body temperature information and location information received from the user terminal 200A through the manager web screens.

A manager web screen shown in FIG. 6 may be a screen for managing the patch-type thermometer 100A. For example, the screen for managing the patch-type thermometer 100A may include model names, model identifiers (IDs), names, states of use, user identification numbers, elapsed times, etc.

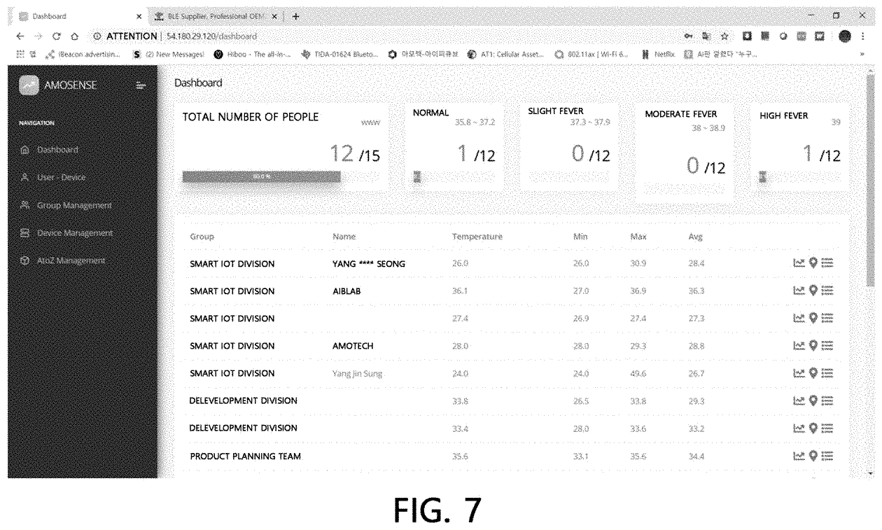

A manager web screen shown in FIG. 7 may be a screen for managing a current monitoring situation. For example, the screen for managing a current monitoring situation may include the number of normal cases, cases having a slight fever, cases having a moderate fever, cases having a high fever, etc. compared to a total number of people and individual-specific body temperature information. Here, the body temperature information may include a current body temperature, a minimum body temperature, a maximum body temperature, and an average body temperature.

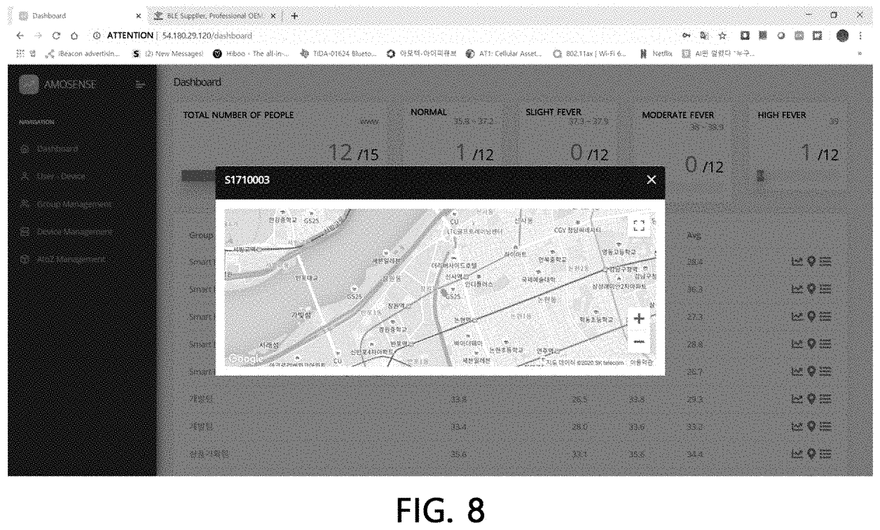

A manager web screen shown in FIG. 8 may provide travel route information in accordance with location information of a user. For example, the manager web screen may provide individual-specific travel route information so that the individual-specific travel route information is displayed on a map.

A manager web screen shown in FIG. 9 may provide user-specific trends of body temperature changes. For example, the manager web screen may provide the trends of body temperature changes with respect to time in the form of a graph.

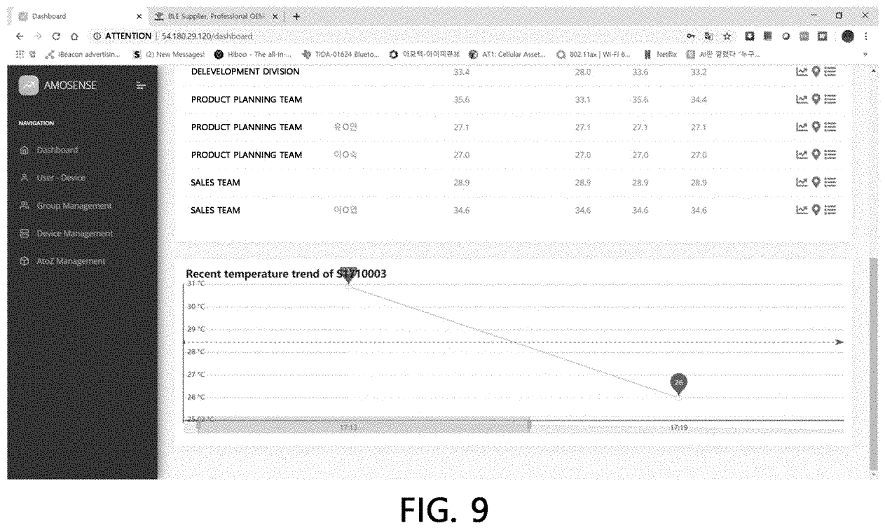

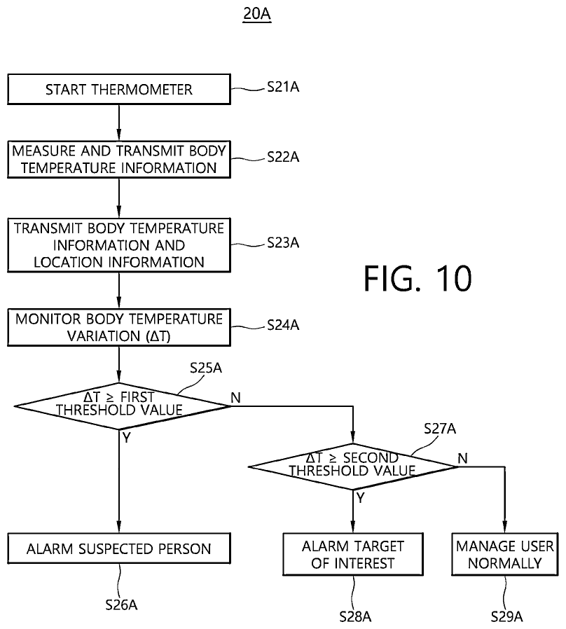

Referring to FIG. 10, a non-face-to-face body temperature change management platform service method 20A according to the first embodiment of the present invention includes a thermometer starting step S21A, a body temperature information measurement and transmission step S22A, a body temperature information and location information transmission step S23A and body temperature variation (Δ) monitoring steps S24A to S29A. In other words, S21A to S23A are included in a first step, and S24A to S29A are included in a second step.

More specifically, the user terminal 200A is tapped on the patch-type thermometer 100A attached to the user to start the patch-type thermometer 100A (S21A). When the user terminal 200A is tapped on the patch-type thermometer 100A, the user terminal 200A may form a magnetic field. At the same time, the patch-type thermometer 100A may generate power in an energy harvesting manner based on inductive coupling with the magnetic field formed from the user terminal 200A. Accordingly, the patch-type thermometer 100A may start operating.

Subsequently, the patch-type thermometer 100A measures and transmits a body temperature to the user terminal 200A in response to a request from the user terminal 200A (S22A). For example, the patch-type thermometer 100A may hourly transmit body temperature information that is obtained through self-measurement by tapping the user terminal 200A on the patch-type thermometer 100A to the user terminal 200A on the basis of NFC.

Optionally, the patch-type thermometer 100A may measure bio-information of the user and transmit the bio-information to the user terminal 200A together with the body temperature information. For example, the patch-type thermometer 100A may measure at least one piece of bio-information among the user's heart rate, electrocardiogram, oxygen saturation, and blood pressure through the detection sensor.

Subsequently, the user terminal 200A transmits the body temperature information and location information to the data server 300A (S23A). Here, the user terminal 200A may receive the body temperature information transmitted by the patch-type thermometer 100A. Also, the user terminal 200A may receive GPS location information from GPS satellites (not shown).

The user terminal 200A may transmit the body temperature information and the location information at different periods. As an example, the user terminal 200A may transmit the location information to the data server 300A at shorter periods than the body temperature information. As another example, the user terminal 200A may collect the location information at shorter periods than the body temperature information and simultaneously transmit the location information together with the body temperature information.

Subsequently, the data server 300A receives the body temperature information and the location information of the user from the user terminal 200A and monitors the body temperature variation Δ (S24A). Here, the body temperature variation Δ is the amount of change in the body temperature compared to an average body temperature of the user. Also, the data server 300A may track the location of the user and find a travel route of the user in accordance with the location information.

An application dedicated to body temperature change and location monitoring may be installed on the user terminal or the manager terminal 200′A. Accordingly, the user terminal or the manager terminal 200′A may receive results of monitoring the body temperature variation Δ from the data server 300 through the dedicated application.

Also, the data server 300A monitors and classifies the body temperature variation Δ as a case of a first threshold value or more, a case of a second threshold value or more and less than the first threshold value, or a case of less than the second threshold value

More specifically, the data server 300A determines whether the body temperature variation Δ of the user is the case of the first threshold value or more (S25A), and when the body temperature variation Δ is the case of the first threshold value or more, the data server 300A may determine the user to be a suspected person. Here, the first threshold value may be a body temperature value from which a change in the body temperature may be determined to be a fever. In other words, when the user is suspected to have a fever in accordance with a change in the body temperature, the data server 300A may determine the user to be a suspected person.

In this case, the data server 300A alarms the integrated control server 400A, the user terminal 200A, and the manager terminal with the corresponding fact (S26A). Here, the integrated control server 400A may include the server of the center for disease control and prevention and the disinfection manager monitoring server.

When it is determined in S25A that the body temperature variation Δ is the case of less than the first threshold value, it is determined whether the body temperature variation Δ is the second threshold value or more (S27A). When the body temperature variation Δ is the second threshold value or more, that is, when the body temperature variation Δ is the case of the second threshold value or more and less than the first threshold value, the user may be determined as a target of interest. Here, the second threshold value may be smaller than the first threshold value. In other words, when the user does not have a fever but his or her body temperature variation is relatively large, the data server 300A may determine the user to be a target of interest.

In this case, the data server 300A alarms the integrated control server 400A, the user terminal 200A, and the manager terminal with the corresponding fact (S28A).

When it is determined in S27A that the body temperature variation Δ is the case of less than the second threshold value, the data server 300A determines the user to be a normal user and manages the user normally (S29A). Here, the second threshold value may be a body temperature value corresponding to a usual body temperature change of each individual. For example, the second threshold value may be a temporary body temperature change caused by fast walking, exercise, etc.

The above-described methods may be implemented by the patch-type thermometer 100A, the user terminal 200A, and the data server 300A shown in FIG. 1.

### Second Embodiment

A non-face-to-face body temperature change management platform service system 10B according to the second embodiment of the present invention is a platform service system for periodically transmitting body temperature information measured through the patch-type thermometer 100B for the purpose of management and may automatically transmit body temperature information measured from a user to the data server 400B through a beacon gateway 200B so that changes in the body temperature of the user can be managed.

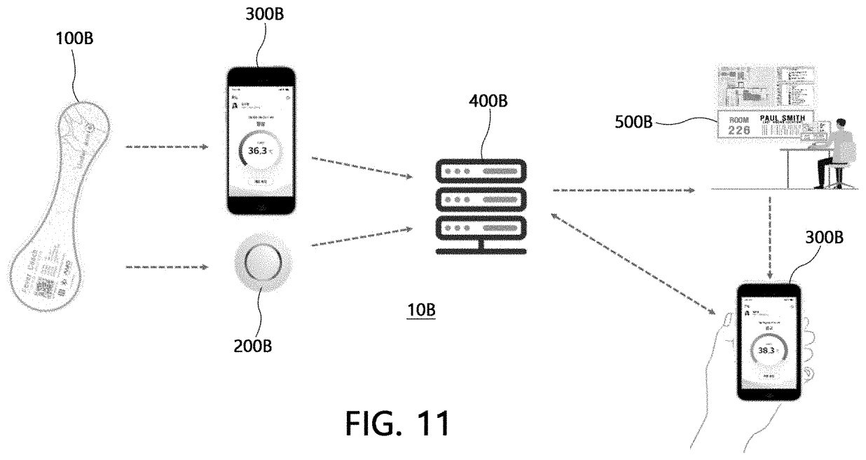

To this end, the non-face-to-face body temperature change management platform service system 10B according to the second embodiment of the present invention includes the patch-type thermometer 100B, the beacon gateway 200B, and the data server 400A as shown in FIG. 11.

The patch-type thermometer 100B may periodically measure the body temperature while attached to the user's body and then store the measurement result. Here, the patch-type thermometer 100B may measure the body temperature at certain time intervals and perform advertising transmission of the body temperature at certain periods. The advertising transmission is that the patch-type thermometer 100B transmits information to any receiver module without a pairing operation or the like of specifying a receiver module. Here, the transmitted information may be encrypted and decrypted. For example, the patch-type thermometer 100B may measure the body temperature every 30 minutes and perform advertising transmission of the body temperature at 2-second time intervals within 30 minutes. Accordingly, the user or the manager can readily check the trend of change in the body temperature of the user by readily checking the stored body temperature information.

The beacon gateway 200B may receive the body temperature information transmitted by the patch-type thermometer 100B and transmit the body temperature information to the data server 400B. Here, the beacon gateway 200B may be installed in a predetermined place.

In this way, the non-face-to-face body temperature change management platform service system 10B according to the second embodiment of the present invention can automatically transmit body temperature information to the data server 400B through the beacon gateway 200B without any specific manipulation by the user as long as the patch-type thermometer 100B is attached. Accordingly, changes in the body temperature can be effectively monitored.

Also, the beacon gateway 200B may provide corresponding location information. Here, the corresponding location information may be fixed location information of the place in which the beacon gateway 200B is installed. For example, the beacon gateway 200B may provide fixed location information for each installation place in each indoor floor and each indoor space. The beacon gateway 200B may transmit the received body temperature information and the information on the location at which the beacon gateway 200B is installed to the data server 400B together.

In this way, the non-face-to-face body temperature change management platform service system 10B according to the second embodiment of the present invention can have the above-described first advantage.

The data server 400B may receive the body temperature information and the location information of the user from the beacon gateway 200B and manage the received information. However, operations and effects of the data server 400B are the same as described above about the data server 300A in the first embodiment except that the reference numeral of each element is changed and the user terminal 200A is replaced with the beacon gateway 200B.

Meanwhile, the non-face-to-face body temperature change management platform service system 10B may further include a user terminal 300B and an integrated control server 500B.

The user terminal 300B may serve as an auxiliary device to the beacon gateway 200B. In other words, the user terminal 300B may receive the body temperature information transmitted by the patch-type thermometer 100B and transmit the body temperature information to the data server 400B. For example, the user terminal 300B may be a portable electronic device such as a smartphone.

Also, the user terminal 300B may collect GPS location information from GPS satellites (not shown) and transmit the GPS location information to the data server 400B. Here, the user terminal 300B may include a GPS receiver unit. As an example, the user terminal 300B may transmit location information at shorter periods than body temperature information. As another example, the user terminal 300B may collect location information at shorter periods than body temperature information and simultaneously transmit the location information together with the body temperature information.

An application dedicated to body temperature change and location monitoring may be installed on the user terminal 300B. Here, the user terminal 300B may receive monitoring results regarding the body temperature variation Δ from the data server 400B through the dedicated application. For example, the user terminal 300B may receive a measured body temperature, the trend of changes in the body temperature, a state suspected of a fever, etc. from the data server 400B.

In this way, the non-face-to-face body temperature change management platform service system 10B according to the second embodiment of the present invention can have the above-described second advantage.

FIG. 11 shows that that the user terminal 300B transmits body temperature information and a manager terminal 300′B receives monitoring results from the data server 400B through the dedicated application.

Operations and effects of the integrated control server 500B are the same as described above about the integrated control server 400A in the first embodiment except that the reference numeral of each element is changed.

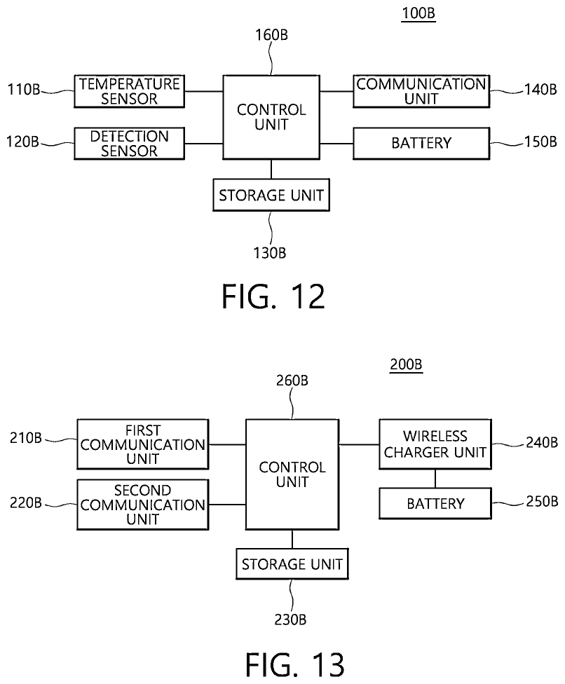

Referring to FIG. 12, the patch-type thermometer 100B is manufactured on the basis of a flexible circuit board and may include a temperature sensor 110B, a storage unit 130B, a communication unit 140B, a battery 150B, and a control unit 160B.

The temperature sensor 110B is the same as described above about the temperature sensor 110A in FIG. 2 except that the reference numeral of each element is changed.

The storage unit 130B may temporarily store body temperature information measured by the temperature sensor 110B. The storage unit 130B may be integrated with the control unit 160B.

The communication unit 140B may transmit the stored body temperature information to the beacon gateway 200B using a short-range communication method. For example, the communication unit 140B may perform communication using Bluetooth Low Energy (BLE) or NFC. Particularly, in the case of Bluetooth, the communication unit 140B may be paired with the beacon gateway 200B or the user terminal 300B. However, the communication unit 140B is not limited thereto and may transmit information, such as the body temperature and the like, without pairing.

The battery 150B supplies power to the patch-type thermometer 100B and may be an ordinary primary battery such as a coin battery, a paper battery, a printed battery, etc. The battery 150B may be provided as a secondary battery that is rechargeable when necessary. Accordingly, the patch-type thermometer 100B can be reduced in overall weight while ensuring sufficient power for operating and also can be implemented in a thin form.

The control unit 160B may control the above-described overall operations of the patch-type thermometer 100B in communication with the temperature sensor 110B, the storage unit 130B, the communication unit 130B, and the battery 150B. For example, the control unit 160B may be a microprocessor.

Meanwhile, the patch-type thermometer 100B may further include a detection sensor 120B for measuring bio-information of the user. Here, the bio-information is the same as described in the first embodiment.

The bio-information may be stored in the storage unit 130B. Accordingly, the patch-type thermometer 100B may transmit the measured bio-information to the beacon gateway 200B or the user terminal 300B together with the stored body temperature information.

In this way, the non-face-to-face body temperature change management platform service system 10B according to the second embodiment of the present invention can have the above-described third advantage.

Referring to FIG. 13, the beacon gateway 200B may include a first communication unit 210B, a second communication unit 220B, a storage unit 230B, a battery 250B, and a control unit 260B.

The first communication unit 210B may perform short-range wireless communication with the patch-type thermometer 100B. For example, the first communication unit 210B may perform wireless communication using Bluetooth or NFC.

The second communication unit 220B may perform long-range wireless communication with the data server 400B. For example, the second communication unit 220B may communicate with the data server 400B through a public communication network such as a wired Internet connection and/or WiFi.

The storage unit 230B may store the body temperature information and location information received from the patch-type thermometer 100B. Here, the storage unit 230B may store the body temperature information and the location information for a preset time period. In other words, the storage unit 230B may store the body temperature information and the location information for a certain time even after the body temperature information and the location information is transmitted to the data server 400B.

The beacon gateway 200B may be supplied with power from a wall electrical outlet or the embedded battery 250B. In this case, the battery 250B may further include a wireless charging unit 240B that is supplied with power through a wireless power transmission module which is not shown but is connected to the wall electrical outlet.

The control unit 260B may control the above-described overall operations of the beacon gateway 200B in communication with the first communication unit 210B, the second communication unit 220B, the storage unit 230B, and the battery 250B. For example, the control unit 260B may be a microprocessor.

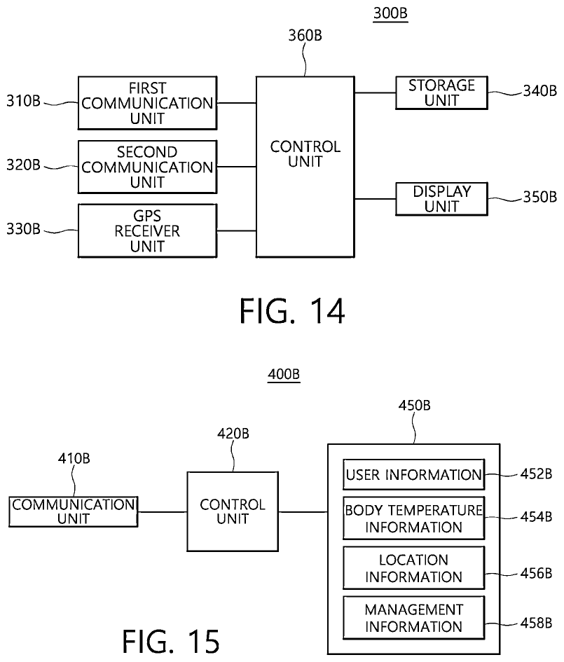

Referring to FIG. 14, the user terminal 300B may include a first communication unit 310B, a second communication unit 320B, a GPS receiver unit 330B, a storage unit 340B, a display unit 350B, and a control unit 360B.

The first communication unit 310B may perform short-range wireless communication with the patch-type thermometer 100B. For example, the first communication unit 310B may perform wireless communication using Bluetooth or NFC.

The second communication unit 320B, the GPS receiver unit 330B, the storage unit 340B, the display unit 350B, and the control unit 360B are the same as described above about the second communication unit 220A, the GPS receiver unit 230A, the storage unit 240A, the display unit 250A, and the control unit 260A in FIG. 3 except that the reference numeral of each element is changed.

Referring to FIG. 15, the data server 400B may include a communication unit 410B, a control unit 420B, and a database 450B.

The communication unit 410B may perform long-range wireless communication with the beacon gateway 200B or the user terminal 300B. For example, the communication unit 410B may communicate with the beacon gateway 200B or the user terminal 200B through a public communication network.

The control unit 420B, the database 450B, user information 452B, body temperature information 454B, and management information 458B may be the same as described above about the control unit 320A, the database 350A, the user information 352A, the body temperature information 354B, and the management information 358B in FIG. 4 except that the reference numeral of each element is changed.

Location information 456B may be location information received from the beacon gateway 200B on the basis of installation location information. Also, the location information 456B may be GPS location information received from the user terminal 300B. The location information 456B may include time-specific location information.

Referring to FIGS. 6 to 9, the data server 400B may readily manage body temperature information and location information received from the beacon gateway 200B and the user terminal 300B through the manager web screens.

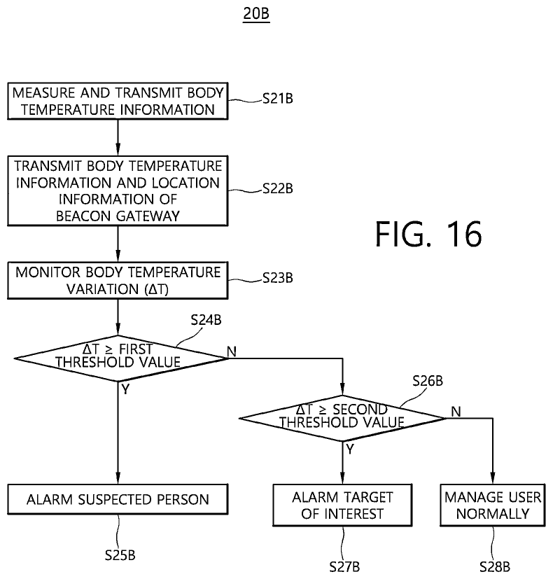

The manager web screen shown in FIG. 6 may be a screen for managing the patch-type thermometer 100B. However, exemplary description of the manager web screen according to the second embodiment is the same as the above exemplary description of the manager web screens of FIGS. 6 to 9 according to the first embodiment. Referring to FIG. 16, a non-face-to-face body temperature change management platform service method 20B according to the second embodiment of the present invention includes a body temperature information measurement and transmission step S21B, a body temperature information and beacon gateway location information transmission step S22B, and body temperature variation (Δ) monitoring steps S23B to S28B. In other words, S21B and S22B are included in a first step, and S23B to S28B are included in a second step.

More specifically, the patch-type thermometer 100B periodically measures and transmits a body temperature of a user to the beacon gateway 200B (S21B). Here, the patch-type thermometer 100B may measure the body temperature at certain time intervals and perform advertising transmission of the body temperature at certain periods. For example, the patch-type thermometer 100B may measure the body temperature every 30 minutes and perform advertising transmission of the body temperature at 2-second time intervals within 30 minutes.

Optionally, the patch-type thermometer 100B may measure bio-information of the user and transmit the bio-information to the beacon gateway 200B together with the body temperature information. For example, the patch-type thermometer 100B may measure at least one piece of bio-information among the user's heart rate, electrocardiogram, oxygen saturation, and blood pressure through the detection sensor.

Subsequently, the beacon gateway 200B transmits the body temperature information and location information thereof to the data server 400B (S22B). The data server 400B may receive the body temperature information transmitted by the patch-type thermometer 100B. Here, the beacon gateway 200B may be installed in a predetermined place. Also, the location information may be fixed location information of the place in which the beacon gateway 200B is installed. For example, the beacon gateway 200B may provide fixed location information for each installation place in each indoor floor and each indoor space.

Optionally, the user terminal 300B may transmit the body temperature information and the location information to the data server 400B. Here, the user terminal 300B may receive the body temperature information transmitted by the patch-type thermometer 100B. Also, the user terminal 300B may receive GPS location information from GSP satellites (not shown).

Here, the user terminal 300B may transmit the body temperature information and the location information at different periods. As an example, the user terminal 300B may transmit the location information to the data server 400B at shorter periods than the body temperature information. As another example, the user terminal 300B may collect the location information at shorter periods than the body temperature information and simultaneously transmit the location information together with the body temperature information.

Subsequently, the data server 400B receives the body temperature information and the location information of the user from the beacon gateway 200B to monitor the body temperature variation Δ (S23B).

S23B to S28B are the same as described above about S24A to S29A in FIG. 10 except that the reference numeral of each element is changed.

The above methods may be implemented by the patch-type thermometer 100B, the beacon gateway 200B, the user terminal 300B, and the data server 400B shown in FIG. 11.

### Third Embodiment

A non-face-to-face body temperature change management platform service system 10C according to the third embodiment of the present invention is a platform service system for periodically transmitting body temperature information measured through a patch-type thermometer 100C for the purpose of management and may automatically transmit body temperature information measured from a user to the data server 400C through a location tracker 200C so that changes in the body temperature of the user can be managed. The location tracker 200C may be a dedicated Internet of Things (IoT) device.

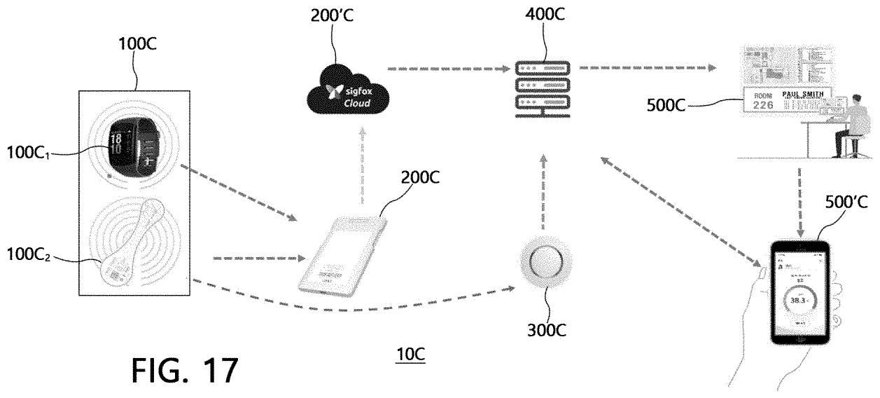

To this end, the non-face-to-face body temperature change management platform service system 10C according to the third embodiment of the present invention includes the patch-type thermometer 100C, the location tracker 200C, and the data server 400C as shown in FIG. 17.

The thermometer 100C may periodically measure and transmit the body temperature of the user. For example, the thermometer 100C may be any one of a band-type thermometer 100C₁ worn on the user's wrist and a patch-type thermometer 100C₂ attached to the user's body in accordance with the form of use.

The band-type thermometer 100C₁ may be implemented in the form of a band and worn on the user's wrist. Here, the band-type thermometer 100C₁ may include a rechargeable battery. In other words, the band-type thermometer 100C₁ may be permanently used compared to the patch-type thermometer 100C₂. Accordingly, unlike the patch-type thermometer 100C₂, it is unnecessary to frequently replace the band-type thermometer 100C₁, and thus convenience of the user can be improved.

For example, the patch-type thermometer 100C₂ may be attached to the armpit or waist of the user's body. When the patch-type thermometer 100C₂ is attached to the waist, it is less likely to fall from the body due to the user's sweat or movement, and thus the patch-type thermometer 100C₂ may accurately measure the body temperature. Accordingly, the data server 400C can monitor the body temperature variation Δ more stably.

While the thermometer 100C is attached to the user's body or worn on the user's wrist, the thermometer 100C may periodically measure the body temperature and then store the measurement result. Here, the thermometer 100C may measure the body temperature at certain time intervals and perform advertising transmission of the body temperature at certain periods. The advertising transmission is that the thermometer 100C transmits information to any receiver module without a pairing operation or the like of specifying a receiver module. Here, the transmitted information may be encrypted and decrypted. For example, the thermometer 100C may measure the body temperature every 30 minutes and perform advertising transmission of the body temperature at 2-second time intervals within 30 minutes. Accordingly, a manager can readily check the trend of change in the body temperature of the user by readily checking the stored body temperature information.

The location tracker 200C is worn by the user and may receive the body temperature information transmitted by the thermometer 100C and transmit the body temperature information to the data server 400C. For example, the location tracker 200C is intended to identify a user and a location thereof when the user accesses a specific facility, and may be an access pass, an employee identification card, or a visitor pass according to the purpose of use. Here, the specific facility is a facility that may accommodate a large number of people such as a workplace, a building, a factory, a school, a theater, an amusement park, a hospital, etc.

In this way, the non-face-to-face body temperature change management platform service system 10C according to the third embodiment of the present invention can transmit body temperature information through the location tracker 200C. Accordingly, it is possible to effectively manage changes in the body temperature of a user who does not carry a personal portable terminal or whose personal portable terminal is prevented from being used.

Also, the location tracker 200C may collect GPS location information from GPS satellites (not shown) and transmit the GPS location information to the data server 400C. Here, the location tracker 200C may include a GPS receiver unit. As an example, the location tracker 200C may transmit the location information at shorter periods than the body temperature information. As another example, the location tracker 200C may collect the location information at shorter periods than the body temperature information and simultaneously transmit the location information together with the body temperature information.

In this way, the non-face-to-face body temperature change management platform service system 10C according to the third embodiment of the present invention can have the above-described first advantage.

The location tracker 200C may transmit the body temperature information and the location information of the user to the data server 400C through a Sigfox IoT server 200′C. For example, the location tracker 200C may upload the body temperature information and the location information to the data server 400C on the basis of a unidirectional low-power wide area network (LPWAN). Here, the Sigfox IoT server 200′C may be a server for running/managing the LPWAN.

In this way, the non-face-to-face body temperature change management platform service system 10C according to the third embodiment of the present invention can minimize power consumption of the location tracker 200C on the basis of unidirectional transmission of a small amount of data. Accordingly, the location tracker 200C can be used for a long time, and it is possible to reduce maintenance costs for battery charging, battery replacement, etc.

The data server 400C may receive the body temperature information and the location information of the user from the location tracker 200C through the Sigfox IoT server 200′C and manage the received information. However, operations and effects of the data server 400C are the same as described above about the data server 300A in the first embodiment except that the reference numeral of each element is changed, the patch-type thermometer 100A is replaced with the thermometer 100C, and the user terminal 200A is replaced with the location tracker 200C.

Here, the data server 400C may alarm an integrated control server 500C, such as control headquarters installed in a center for disease control and prevention, companies, hospitals, etc., and a manager terminal 500′C with a determination result (fact) on a suspected person or a target of interest.

Meanwhile, the non-face-to-face body temperature change management platform service system 10C may further include a beacon gateway 300C and the integrated control server 500C.

Operations and effects of the beacon gateway 300C are the same as described above about the beacon gateway 200B in the second embodiment except that the reference numeral of each element is changed and the patch-type thermometer 100A is replaced with the thermometer 100C.

Here, the beacon gateway 300C functions better indoors, and the location tracker 200C functions better outdoors. Accordingly, it is possible to provide accurate location information of the user.

Operations and effects of the integrated control server 500C are the same as described above about the integrated control server 400A in the first embodiment except that the reference numeral of each element is changed.

However, the integrated control server 500C may transmit a precise examination request, an isolation request, etc. to the manager terminal 500′C through an emergency message or a phone call. Here, the manager terminal 500′C is the same as described above about the manager terminal 200′A in the first embodiment except that the reference numeral of each element is changed.

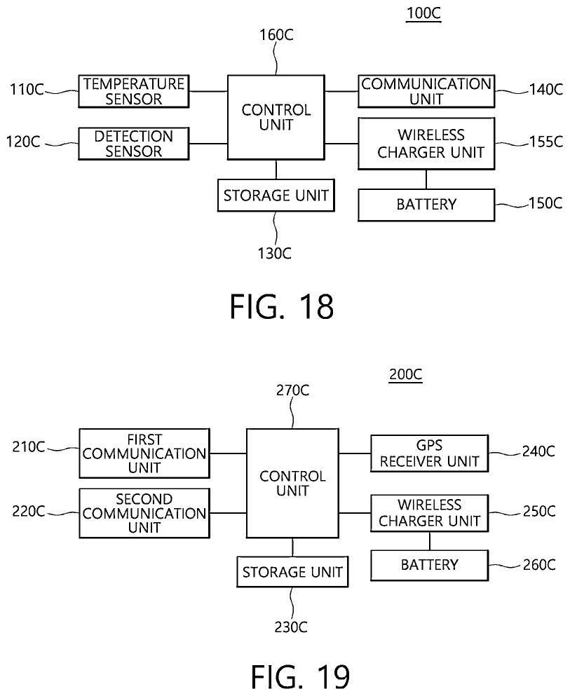

Referring to FIG. 18, the thermometer 100C may include a temperature sensor 110C, a storage unit 130C, a communication unit 130C, a battery 140C, and a control unit 150C. Here, the patch-type thermometer 100C2 may be manufactured on the basis of a flexible circuit board.

The temperature sensor 110C is the same as described above about the temperature sensor 110A in FIG. 2 except that the reference numeral of each element is changed.

The storage unit 130C is the same as described above about the storage unit 130C in FIG. 12 except that the reference numeral of each element is changed.

The communication unit 130C may transmit the stored body temperature information to the location tracker 200C or the beacon gateway 300C using a short-range communication method. For example, the communication unit 130C may perform communication using BLE or NFC. Particularly, in the case of Bluetooth, the communication unit 130B may be paired with the location tracker 200C or the beacon gateway 300C. However, the communication unit 140C is not limited thereto and may transmit information, such as the body temperature and the like, without pairing.

The battery 140C supplies power to the thermometer 100C. Here, the patch-type thermometer 100C₂ may be an ordinary primary battery such as a coin battery, a paper battery, a printed battery, etc. Accordingly, the patch-type thermometer 100C₂ can be reduced in overall weight while ensuring sufficient power for operating and also can be implemented in a thin form. Also, the band-type thermometer 100C₁ may be provided as a secondary battery that is rechargeable.

The control unit 150C may control the above-described overall operations of the thermometer 100C in communication with the temperature sensor 110C, the storage unit 130C, the communication unit 130C, and the battery 140C. For example, the control unit 150C may be a microprocessor.

Meanwhile, the thermometer 100C may further include a detection sensor 120C and a wireless charger unit 145. Here, the wireless charger unit 145 may be included in the band-15 type thermometer 100C₁.

The detection sensor 120C may measure bio-information of the user. Here, the bio-information is the same as described in the first embodiment.

The bio-information may be stored in the storage unit 130C. Accordingly, the thermometer 100C may transmit the measured bio-information to the location tracker 200C or the beacon gateway 300C together with the stored body temperature information.

In this way, the non-face-to-face body temperature change management platform service system 10C according to the third embodiment of the present invention can have the above-described third advantage.

The wireless charger unit 145 may receive power from an external power source through a wireless power transmission module (not shown) and wirelessly charge the battery 140C. Here, the thermometer 100C may further include a wireless charging cradle. Alternatively, the wireless charger unit 145 may be connected to the external power source through a wire to charge the battery 140C.

Accordingly, the thermometer 100C may permanently used through charging, and thus economic benefits of the user can be increased.

Referring to FIG. 19, the location tracker 200C may include a first communication unit 210C, a second communication unit 220C, a storage unit 230C, a GPS receiver unit 230, a battery 260C, and a control unit 270C.

The first communication unit 210C may perform short-range wireless communication with the thermometer 100C. For example, the first communication unit 210C may perform wireless communication using Bluetooth or NFC.

The second communication unit 220C may upload the body temperature information and the location information to the Sigfox IoT server 200′C. Here, the second communication unit 220C may perform one-way communication to the Sigfox IoT server 200′C.

The storage unit 230C may store the body temperature information and the location information received from the thermometer 100C. Here, the storage unit 230C may store the body temperature information and the location information for a preset time period. In other words, the storage unit 230C may store the body temperature information and the location information for a certain time even after the body temperature information and the location information is transmitted to the Sigfox IoT server 200′C.

The GPS receiver unit 230 may receive location information from the GPS satellites. The GPS receiver unit 230 may store the received location information in the storage unit 230C. The GPS receiver unit 230 may store time-specific location information to transmit later the location information to the Sigfox IoT server 200′C at once.

The battery 260C supplies power to the location tracker 200C and may be a coin battery or a polygon battery. Alternatively, the battery 260C may be a flat flexible battery for reducing overall weight and thickness. In this case, the battery 260C may be a secondary battery that is rechargeable.

The control unit 270C may control the above-described overall operations of the location tracker 200C in communication with the first communication unit 210C, the second communication unit 220C, the storage unit 230C, the GPS receiver unit 230, and the battery 260C. For example, the control unit 270C may be a microprocessor.

Meanwhile, the location tracker 200C may further include the wireless charger unit 240C.

The wireless charger unit 240C may receive power from an external power source through a wireless power transmission module (not shown) and wirelessly charge the battery 260C. Here, the location tracker 200C may further include a wireless charging cradle. Alternatively, the wireless charger unit 240C may be connected to the external power source through a wire to charge the battery 260C.

In this way, the non-face-to-face body temperature change management platform service system 10C according to the third embodiment of the present invention can be repeatedly used through charging, and thus only the patch-type thermometer 100C₂ can be replaced and used again. Accordingly, maintenance costs can be minimized, and it is possible to improve economic benefits and manageability.

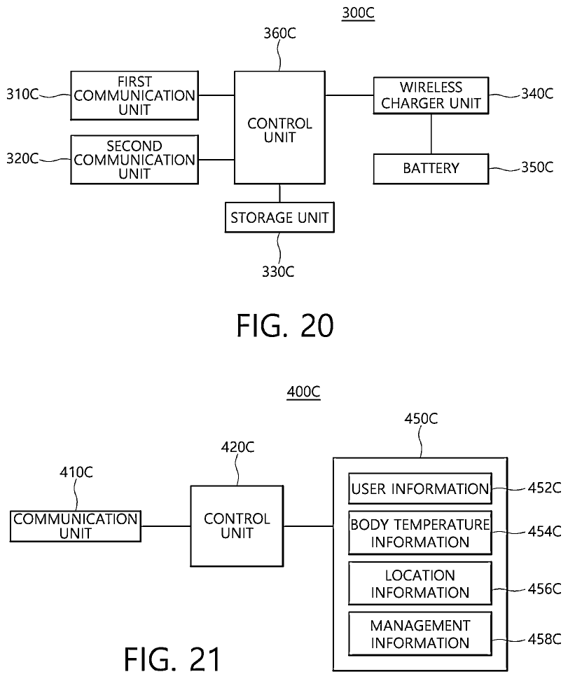

Referring to FIG. 20, the beacon gateway 300C may include a first communication unit 310C, a second communication unit 320C, a storage unit 330C, a battery 350C, and a control unit 360C.

Also, the beacon gateway 300C may further include a wireless charger unit 340C. The first communication unit 310C, the second communication unit 320C, the storage unit 330C, the wireless charger unit 340C, the battery 350C, and the control unit 360C are the same as described above about the first communication unit 210B, the second communication unit 220B, the storage unit 230B, the wireless charger unit 240C, the battery 250B, and the control unit 260C in FIG. 13 except that the reference numeral of each element is changed and the patch-type thermometer 100B is replaced with the thermometer 100C.

Referring to FIG. 21, the data server 400C may include a communication unit 410C, a control unit 420C, and a database 450C.

The communication unit 410C may perform long-range wireless communication with the Sigfox IoT server 200′C or the beacon gateway 300C. For example, the communication unit 410C may communicate with the Sigfox IoT server 200′C or the beacon gateway 300C through a public communication network.

The control unit 420C, the database 450C, user information 452C, and management information 458C may be the same as described above about the control unit 320A, the database 350A, the user information 352A, and the management information 358B in FIG. 4 except that the reference numeral of each element is changed and the patch-type thermometer 100B is replaced with the thermometer 100C.

Body temperature information 454C may be body temperature information measured by the thermometer 100C and transmitted through the Sigfox IoT server 200′C or the beacon gateway 300C. Here, the body temperature information may include the trend of period-specific body temperature variations Δ.

Location information 456C may be GPS location information received from the location tracker 200C. Also, the location information 456C may be location information received from the beacon gateway 300C on the basis of installation location information. The location information 456C may include time-specific location information.

Referring to FIGS. 6 to 9, the data server 400C may readily manage the location tracker 200C and body temperature information and location information received from the location tracker 200C through the manager web screens.

The manager web screen shown in FIG. 6 may be a screen for managing the thermometer 100C. However, exemplary description of the manager web screen according to the third embodiment is the same as the above exemplary description of the manager web screens of FIGS. 6 to 9 according to the first embodiment.

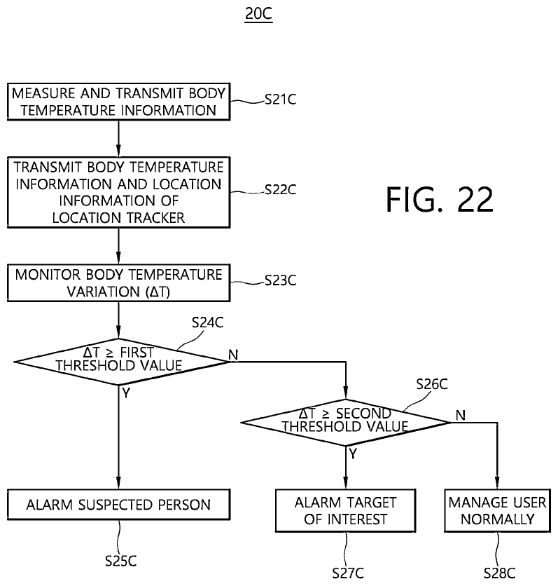

Referring to FIG. 22, a non-face-to-face body temperature change management platform service method 20C according to the third embodiment of the present invention includes a body temperature information measurement and transmission step S21C, a body temperature information and location tracker location information transmission step S22C, and body temperature variation (Δ) monitoring steps S23C to S28C. In other words, S21C and S22C are included in a first step, and S23C to S28C are included in a second step.

More specifically, the thermometer 100C periodically measures and transmits a body temperature of a user to the location tracker 200C (S21C). Here, the thermometer 100C may measure the body temperature at certain time intervals and perform advertising transmission of the body temperature at certain periods. For example, the thermometer 100C may measure the body temperature every 30 minutes and perform advertising transmission of the body temperature at 2-second time intervals within 30 minutes.

Optionally, the thermometer 100C may measure bio-information of the user and transmit the bio-information to the location tracker 200C together with the body temperature information. For example, the thermometer 100C may measure at least one piece of bio-information among the user's heart rate, electrocardiogram, oxygen saturation, and blood pressure through the detection sensor.

Subsequently, the location tracker 200C worn by the user transmits the body temperature information and location information thereof to the data server 400C through the Sigfox IoT server 200′C (S22C). Here, the location tracker 200C may upload the body temperature information and the location information to the data server 400C on the basis of a unidirectional LPWAN.

The location tracker 200C may receive the body temperature information transmitted by the thermometer 100C. Also, the location tracker 200C may receive GPS location information from GSP satellites (not shown).

Here, the location tracker 200C may transmit the body temperature information and the location information at different periods. As an example, the location tracker 200C may transmit the location information to the data server 400C through the Sigfox IoT server 200′C at shorter periods than the body temperature information. As another example, the location tracker 200C may collect the location information at shorter periods than the body temperature information and simultaneously transmit the location information together with the body temperature information.

Optionally, the beacon gateway 300C may transmit the body temperature information and the location information to the data server 400C. Here, the beacon gateway 300C may receive the body temperature information transmitted by the thermometer 100C. Here, the beacon gateway 300C may be installed in a predetermined place. Also, the location information may be fixed location information of the place in which the beacon gateway 300C is installed. For example, the beacon gateway 300C may provide fixed location information for each installation place in each indoor floor and each indoor space.

Subsequently, the data server 400C receives the body temperature information and the location information of the user from the Sigfox IoT server 200′C or the beacon gateway 300C to monitor the body temperature variation Δ (S23C).

An application dedicated to body temperature change and location monitoring may be installed on the manager terminal 500′C. Accordingly, the manager terminal 500′C may receive results of monitoring the body temperature variation Δ from the data server 400C through the dedicated application.

S23C to S28C are the same as described above about S24A to S29A in FIG. 10 except that the reference numeral of each element is changed.

In S25C and S27C, however, the data server 400C alarms the integrated control server 500C and the manager terminal 500′C with a determination result (corresponding fact) on a suspected person or a target of interest.

The above methods may be implemented by the thermometer 100C, the location tracker 200C, the beacon gateway 300C, and the data server 400C shown in FIG. 17.

### Fourth Embodiment

A non-face-to-face body temperature change management platform service system 10D according to the fourth embodiment of the present invention is a platform service system for periodically transmitting body temperature information measured through the smart thermometer 100D for the purpose of management and may automatically transmit body temperature information measured from a user to a data server through the smart thermometer 100D so that changes in the body temperature of the user can be managed.

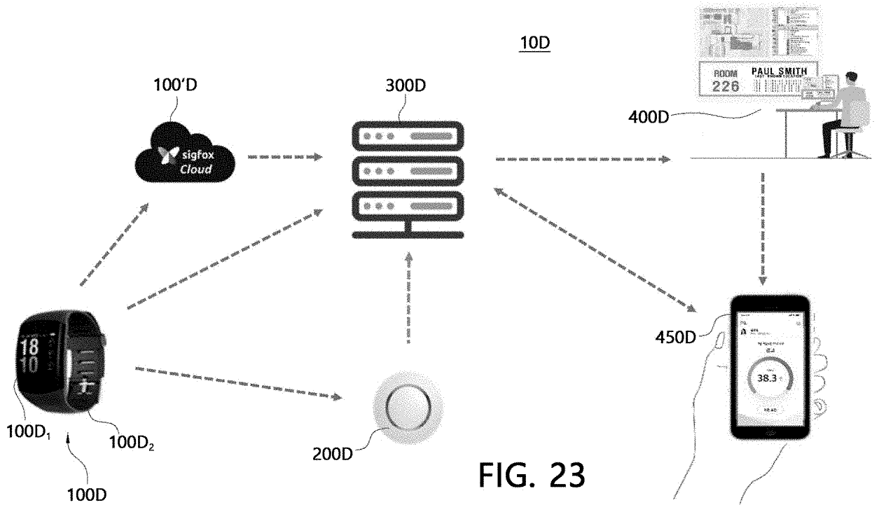

To this end, the non-face-to-face body temperature change management platform service system 10D according to the fourth embodiment of the present invention includes the smart thermometer 100D and the data server 300D as shown in FIG. 23.

The smart thermometer 100D may be implemented in a band-type or a clock-type and worn on the user's wrist. Here, the smart thermometer 100D may include a body 100D₁ and bands 100D₂ provided on the both sides of the body 100D₁. In the smart thermometer 100D, a temperature sensor for measuring a body temperature may be provided in a bottom of the body 100D₁, that is, to be exposed toward the user's wrist.

The smart thermometer 100D worn on the user's wrist may periodically measure and then store the body temperature. Here, the smart thermometer 100D may measure the body temperature at certain time intervals and transmit the body temperature to the data server 300D at certain periods. For example, the smart thermometer 100D may measure the body temperature every 30 minutes.

In this way, the non-face-to-face body temperature change management platform service system 10D according to the fourth embodiment of the present invention can measure and transmit body temperature information without any specific manipulation by the user or any additional device for collecting and transmitting the body temperature as long as the smart thermometer 100D is worn. Accordingly, it is possible to improve convenience in measurement the body temperature and managing changes in the body temperature.

Also, the smart thermometer 100D may collect GPS location information from GPS satellites (not shown) and transmit the GPS location information to the data server 300D. Here, the smart thermometer 100D may include a GPS receiver unit. As an example, the smart thermometer 100D may transmit the location information at shorter periods than the body temperature information. As another example, the smart thermometer 100D may collect the location information at shorter periods than the body temperature information and simultaneously transmit the location information together with the body temperature information.

In this way, the non-face-to-face body temperature change management platform service system 10D according to the fourth embodiment of the present invention can have the above-described first advantage.

Optionally, an application dedicated to body temperature change and location monitoring may be installed on the smart thermometer 100D. Here, the smart thermometer 100D may receive monitoring results regarding a body temperature variation Δ from the data server 300D through the dedicated application. For example, the smart thermometer 100D may receive a measured body temperature, the trend of changes in the body temperature, a state suspected of a fever, etc. from the data server 300D.

The data server 300D may receive the body temperature information and the location information of the user from the smart thermometer 100D and manage the received information. However, operations and effects of the data server 300D are the same as described above about the data server 300A in the first embodiment except that the reference numeral of each element is changed and the patch-type thermometer 100A and the user terminal 200A are replaced with the smart thermometer 100D.

The data server 300D may alarm an integrated control server 400D, such as control headquarters installed in the center for disease control and prevention, companies, hospitals, etc., and a manager terminal 450D with a determination result (fact) on a suspected person or a target of interest.

Meanwhile, the non-face-to-face body temperature change management platform service system 10D may further include a Sigfox IoT server 100′D, a beacon gateway 200D, and the integrated control server 400D.

The Sigfox IoT server 100′D may receive the body temperature information and the location information of the user transmitted by the smart thermometer 100D and transmit the received information to the data server 300D. Here, the Sigfox IoT server 100′D may be a server for running/managing the LPWAN.

The smart thermometer 100D may transmit the body temperature information and the location information of the user to the Sigfox IoT server 100′D, and the data server 300D may receive the body temperature information and the location information of the user from the Sigfox IoT server 100′D. In other words, the smart thermometer 100D may upload the body temperature information and the location information to the data server 300D on the basis of a unidirectional LPWAN.

In this way, the non-face-to-face body temperature change management platform service system 10D according to the fourth embodiment of the present invention can minimize power consumption of the smart thermometer 100D on the basis of a small amount of one-way data transmission, and thus the smart thermometer 100D can be used for a long time. Accordingly, it is possible to minimize frequent operations of battery charging, battery replacement, etc. of the smart thermometer 100D and eliminate a hassle of the user.

Operations and effects of the beacon gateway 200D are the same as described above about the beacon gateway 200B in the second embodiment except that the reference numeral of each element is changed and the patch-type thermometer 100A is replaced with the smart thermometer 100D.

However, the smart thermometer 100D may perform advertising transmission of the body temperature. The advertising transmission is that the smart thermometer 100D transmits information to any receiver module without a pairing operation or the like of specifying a receiver module. Here, the transmitted information may be encrypted and decrypted.

Since the non-face-to-face body temperature change management platform service system 10D according to the fourth embodiment of the present invention can automatically transmit specified indoor location information to the data server 300D together with the body temperature information, changes in the body temperature can be effectively monitored on the basis of precise location information.

Here, the beacon gateway 200D functions better indoors, and the smart thermometer 100D functions better outdoors. Accordingly, it is possible to provide accurate location information of the user.

When the Sigfox IoT server 100′D or the beacon gateway 200D transmits the body temperature information and the location information to the data server 300D, the smart thermometer 100D may measure the body temperature every 30 minutes and transmit the measured body temperature at 2-second time intervals within 30 minutes.

Operations and effects of the integrated control server 400D are the same as described above about the integrated control server 400A in the first embodiment except that the reference numeral of each element is changed.

Also, the integrated control server 400D may transmit a precise examination request, an isolation request, etc. to the manager terminal 450D through an emergency message or a phone call. Here, the manager terminal 450D is the same as described above about the manager terminal 200′A in the first embodiment except that the reference numeral of each element is changed.

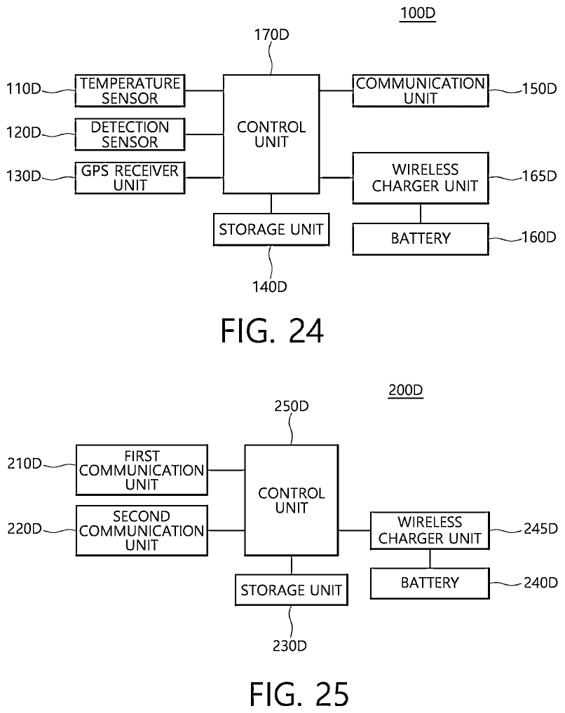

Referring to FIG. 24, the smart thermometer 100D may include a temperature sensor 110D, a GPS receiver unit 130D, a storage unit 140D, a communication unit 150D, a battery 160D, and a control unit 170D.

The temperature sensor 110D may be provided in a bottom of the body 120D and comes into contact with the user's wrist to measure the temperature. Here, the temperature sensor 110D may be a digital temperature sensor. For example, the temperature sensor 110D may be an NTC thermistor.

The GPS receiver unit 130D may receive location information from the GPS satellites. The GPS receiver unit 130D may store the received location information in the storage unit 140D. The GPS receiver unit 130D may store time-specific location information to transmit later the location information to the data server 300D at once.

The storage unit 140D may store the body temperature information and the location information received from the smart thermometer 100D. Here, the storage unit 140D may store the body temperature information and the location information for a preset time period. In other words, the storage unit 140D may store the body temperature information and the location information for a certain time even after the body temperature information and the location information is transmitted to the data server 300D.

The communication unit 150D may perform long-range wireless communication with the data server 300D. As an example, the communication unit 150D may communicate with the data server 300D through a public communication network using a communication protocol such as WiFi, LTE, 5G, etc.

As another example, the communication unit 150D may transmit the body temperature information stored in the storage unit 140D to the beacon gateway 200D using a short-range wireless communication method. For example, the communication unit 150D may perform communication using BLE or NFC. Particularly, in the case of Bluetooth, the communication unit 150D may be paired with the beacon gateway 200D. However, the communication unit 150D is not limited thereto and may transmit information, such as the body temperature and the like, without pairing.

As another example, the communication unit 150D may upload the body temperature information and the location information to the Sigfox IoT server 100′D. Here, the communication unit 150D may perform one-way communication to the Sigfox IoT server 100′D.

The battery 160D supplies power to the smart thermometer 100D. Here, the battery 160D may be a coin battery or a polygon battery. Alternatively, the battery 160D may be a flat flexible battery for reducing overall weight and thickness. In this case, the battery 160D may be a secondary battery that is rechargeable.

The control unit 170D may control the above-described overall operations of the smart thermometer 100D in communication with the temperature sensor 110D, the GPS receiver unit 130D, the storage unit 140D, the communication unit 150D, and the battery 160D. For example, the control unit 170D may be a microprocessor.

Meanwhile, the smart thermometer 100D may further include a detection sensor 120D and a wireless charger unit 165D.

The detection sensor 120D may measure bio-information of the user. Here, the bio-information is the same as described in the first embodiment.

The bio-information may be stored in the storage unit 140D. Accordingly, the smart thermometer 100D may transmit the measured bio-information to the data server 300D together with the body temperature information stored in the storage unit 140D.

In this way, the non-face-to-face body temperature change management platform service system 10D according to the fourth embodiment of the present invention can have the above-described third advantage.

The wireless charger unit 165D may receive power from an external power source through a wireless power transmission module (not shown) and wirelessly charge the battery 160D. Here, the smart thermometer 100D may further include a wireless charging cradle. Alternatively, the wireless charger unit 165D may be connected to the external power source through a wire to charge the battery 160D.

In this way, the non-face-to-face body temperature change management platform service system 10D according to the fourth embodiment of the present invention does not require a preparation operation, such as installing a power code to charge the smart thermometer 100D and the like, and thus convenience of the user can be improved.

Referring to FIG. 25, the beacon gateway 200D may include a first communication unit 210D, a second communication unit 220D, a storage unit 230D, a battery 240D, and a control unit 250D.

Also, the beacon gateway 200D may further include a wireless charger unit 245D. However, the first communication unit 210D, the second communication unit 220D, the storage unit 230D, the battery 240D, the wireless charger unit 245D, and the control unit 250D are the same as described above about the first communication unit 210B, the second communication unit 220B, the storage unit 230B, the wireless charger unit 240C, the battery 250B, and the control unit 260C in FIG. 13 except that the reference numeral of each element is changed and the patch-type thermometer 100B is replaced with the smart thermometer 100D.

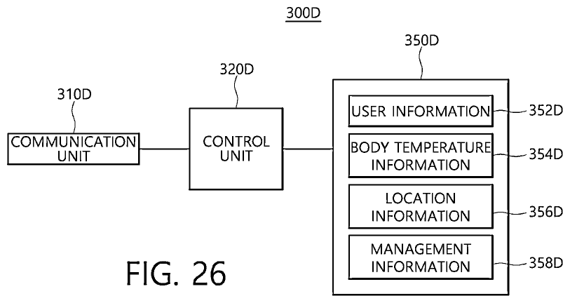

Referring to FIG. 26, the data server 300D may include a communication unit 310D, a control unit 320D, and a database 350.

The communication unit 310D may perform long-range wireless communication with the smart thermometer 100D, the Sigfox IoT server 100′D, or the beacon gateway 200D. For example, the communication unit 310D may communicate with the smart thermometer 100D, the Sigfox IoT server 100′D, or the beacon gateway 200D through a public communication network.

The control unit 320D, the database 350D, user information 352D, and management information 358D may be the same as described above about the control unit 320A, the database 350A, the user information 352A, and the management information 358B in FIG. 4 except that the reference numeral of each element is changed and the patch-type thermometer 100B is replaced with the smart thermometer 100D.

Body temperature information 354D may be body temperature information measured and transmitted by the smart thermometer 100D. Here, the body temperature information may include the trend of period-specific body temperature variations Δ.

Location information 356D may be GPS location information received from the smart thermometer 100D. Also, the location information 356D may be location information received from the beacon gateway 200D on the basis of installation location information. The location information 356D may include time-specific location information.

Referring to FIGS. 6 to 9, the data server 300D may readily manage body temperature information and location information received from the smart thermometer 100D through the manager web screens.

The manager web screen shown in FIG. 6 may be a screen for managing the smart thermometer 100D. However, exemplary description of the manager web screen according to the fourth embodiment is the same as the above exemplary description of the manager web screens of FIGS. 6 to 9 according to the first embodiment.

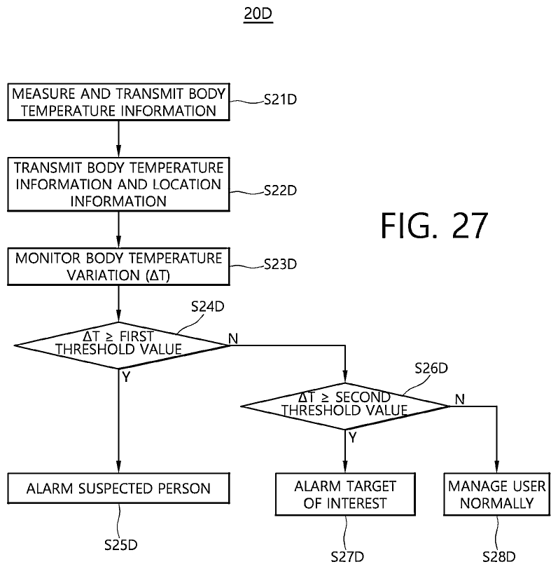

Referring to FIG. 27, a non-face-to-face body temperature change management platform service method 20D according to the fourth embodiment of the present invention includes a body temperature information measurement step S21D, a body temperature information and location information transmission step S22D, and body temperature variation (Δ) monitoring steps S23D to S28D. In other words, S21D and S22D are included in a first step, and S23D to S28D are included in a second step.

More specifically, the band-type smart thermometer 100D worn on the user's wrist periodically measures a body temperature of a user (S21D). Here, the smart thermometer 100D may measure the body temperature at certain time intervals. For example, the smart thermometer 100D may measure the body temperature every 30 minutes.

Optionally, the smart thermometer 100D may measure bio-information of the user. For example, the smart thermometer 100D may measure at least one piece of bio-information among the user's heart rate, electrocardiogram, oxygen saturation, and blood pressure through the detection sensor.

Subsequently, the smart thermometer 100D transmits the body temperature information and location information to the data server 300D (S22D). Here, the smart thermometer 100D may GPS location information from GSP satellites (not shown).

Here, the smart thermometer 100D may transmit the body temperature information and the location information at different periods. As an example, the smart thermometer 100D may transmit the location information to the data server 300D at shorter periods than the body temperature information. As another example, the smart thermometer 100D may collect the location information at shorter periods than the body temperature information and simultaneously transmit the location information together with the body temperature information.

As another example, the beacon gateway 200D may transmit the body temperature information and the location information to the data server 300D. Here, the beacon gateway 200D may receive the body temperature information transmitted by the smart thermometer 100D. Here, the beacon gateway 200D may be installed in a predetermined place. Also, the location information may be fixed location information of the place in which the beacon gateway 200D is installed. For example, the beacon gateway 200D may provide fixed location information for each installation place in each indoor floor and each indoor space.

As still another example, the Sigfox IoT server 100′D may transmit the body temperature information and the location information to the data server 300D. Here, the smart thermometer 100D may transmit the body temperature information and the location information of the user to the Sigfox IoT server 100′D, and the data server 300D may receive the body temperature information and the location information of the user from the Sigfox IoT server 100′D. In other words, the smart thermometer 100D may upload the body temperature information and the location information to the data server 300D on the basis of a unidirectional LPWAN.

When the Sigfox IoT server 100′D or the beacon gateway 200D transmits the body temperature information and the location information to the data server 300D, the smart thermometer 100D may measure the body temperature every 30 minutes and transmit the measured body temperature at 2-second time intervals within 30 minutes.

Subsequently, the data server 300D receives the body temperature information and the location information of the user measured by the smart thermometer 100D to monitor the body temperature variation Δ (S23D). Here, the data server 300D may receive the body temperature information and the location information of the user from at least one of the smart thermometer 100D, the Sigfox IoT server 100′D, and the beacon gateway 200D.

An application dedicated to body temperature change and location monitoring may be installed on the manager terminal 450D. Accordingly, the manager terminal 450D may receive results of monitoring the body temperature variation Δ from the data server 300D through the dedicated application.

S23D to S28D are the same as described above about S24A to S29A in FIG. 10 except that the reference numeral of each element is changed.

In S25D and S27D, however, the data server 400C alarms the integrated control server 400D and the manager terminal 450D with a determination result (corresponding fact) on a suspected person or a target of interest.

The above methods may be implemented by the smart thermometer 100D, the beacon gateway 200D, and the data server 300D shown in FIG. 23.

Although embodiments of the present invention have been described above, the spirit of the present invention is not limited to the embodiments set forth herein. Those of ordinary skill in the art can readily propose other embodiments by adding, changing, removing, etc. components without departing from the spirit of the present invention, and the other embodiments also fall within the scope of the present invention.

### INDUSTRIAL APPLICABILITY

The present invention relates to a non-face-to-face body temperature change management platform service system and method. The present invention can provide a non-face-to-face body temperature change management platform service system and method for monitoring changes in the body temperature of a user and the location in real time and thus has industrial applicability.

1. A non-face-to-face body temperature change management platform service system comprising:
an electronic device that is located near a user and configured to collect and transmit body temperature information of the user and location information thereof; and
a data server configured to receive the body temperature information and the location information from the electronic device and monitor a body temperature variation (Δ) compared to an average body temperature of the user.

2. The non-face-to-face body temperature change management platform service system of claim 1, wherein the data server monitors and classifies the body temperature variation (Δ) as a case of a first threshold value or more, a case of a second threshold value or more and less than the first threshold value, or a case of less than the second threshold value, and
when the body temperature variation (Δ) of the user is the case of the first threshold value or more, the data server determines the user to be a suspected person and alarms an integrated control server and a manager terminal with a corresponding fact.

3. The non-face-to-face body temperature change management platform service system of claim 2, wherein an application dedicated to body temperature change and location monitoring is installed on the manager terminal, and
the manager terminal receives results of monitoring the body temperature variation (Δ) from the data server through the dedicated application.

4. The non-face-to-face body temperature change management platform service system of claim 2, wherein the integrated control server includes a server of a center for disease control and prevention and a disinfection manager monitoring server.

5. The non-face-to-face body temperature change management platform service system of claim 1, further comprising a patch-type thermometer that is attached to the user's body to measure and transmit the body temperature,
wherein the electronic device is a user terminal that is tapped on the patch-type thermometer to form a magnetic field, requests body temperature measurement from the patch-type thermometer to receive the measured temperature information, and collects Global Positioning System (GPS) location information through a GPS receiver unit to transmit the body temperature information and the location information.

6. The non-face-to-face body temperature change management platform service system of claim 5, wherein the patch-type thermometer is driven in an energy harvesting manner based on inductive coupling with the magnetic field.

7. The non-face-to-face body temperature change management platform service system of claim 5, wherein the patch-type thermometer further comprises a detection sensor configured to measure bio-information of the user, and
the detection sensor measures at least one of the user's heart rate, electrocardiogram, oxygen saturation, and blood pressure.

8. The non-face-to-face body temperature change management platform service system of claim 1, further comprising a patch-type thermometer that is attached to the user's body to periodically measure and transmit the body temperature, and
the electronic device is a beacon gateway that is installed in a predetermined place to provide corresponding location information, receives the body temperature information transmitted by the patch-type thermometer, and transmits the body temperature information and the location information together.

9. The non-face-to-face body temperature change management platform service system of claim 8, further comprising a user terminal configured to receive the body temperature information from the patch-type thermometer, collect Global Positioning System (GPS) location information through a GPS receiver unit, and transmit the body temperature information and the GPS location information to the data server.

10. The non-face-to-face body temperature change management platform service system of claim 8, wherein the patch-type thermometer measures the body temperature at certain time intervals and performs advertising transmission of the body temperature at certain periods.

11. The non-face-to-face body temperature change management platform service system of claim 8, wherein the beacon gateway provides fixed location information for each installation place in each indoor floor and each indoor space.

12. The non-face-to-face body temperature change management platform service system of claim 1, wherein the electronic device comprises:
a battery configured to supply power; and
a wireless charger unit configured to charge the battery.

13. The non-face-to-face body temperature change management platform service system of claim 1, further comprising a thermometer configured to periodically measure and transmit the body temperature of the user,
wherein the electronic device is a location tracker that is worn by the user, collects the body temperature information measured by the thermometer, collects Global Positioning System (GPS) location information through a GPS receiver unit, and transmits the body temperature information and the location information, and
the data server receives the body temperature information and the location information of the user from the location tracker through a Sigfox Internet of Things (IoT) server.

14. The non-face-to-face body temperature change management platform service system of claim 13, further comprising a beacon gateway that is installed in a predetermined place to provide corresponding location information, receives the body temperature information transmitted by the thermometer, and transmits the body temperature information and the location information together.

15. The non-face-to-face body temperature change management platform service system of claim 13, wherein the thermometer is any one of a patch-type thermometer attached to the user's body and a band-type thermometer worn on the user's wrist.

16. The non-face-to-face body temperature change management platform service system of claim 1, wherein the electronic device is a band-type smart thermometer that is worn by the user to periodically measure the body temperature, collects Global Positioning System (GPS) location information through a GPS receiver unit, and transmits the measured body temperature information and the location information.

17. The non-face-to-face body temperature change management platform service system of claim 16, wherein the smart thermometer transmits the body temperature information and the location information of the user to a Sigfox Internet of Things (IoT) server, and the data server receives the body temperature information and the location information of the user from the Sigfox IoT server.

18. A non-face-to-face body temperature change management platform service method comprising:
collecting, by an electronic device near a user, body temperature information of the user and location information of the electronic device and transmitting the body temperature information and the location information to a data server; and
receiving, by the data server, the body temperature information and the location information from the electronic device and monitoring a body temperature variation (Δ) compared to an average body temperature of the user.

19. The non-face-to-face body temperature change management platform service method of claim 18, wherein the transmitting of the body temperature information and the location information comprises:
tapping, by a user terminal which is the electronic device, on a patch-type thermometer attached to the user's body and forming a magnetic field to start the patch-type thermometer;
measuring and transmitting, by the patch-type thermometer, the body temperature in response to a request from the user terminal; and
receiving, by the user terminal, the body temperature information transmitted by the patch-type thermometer, collecting Global Positioning System (GPS) location information through a GPS receiver unit, and transmitting the body temperature information and the GPS location information to the data server.

20. The non-face-to-face body temperature change management platform service method of claim 18, wherein the transmitting of the body temperature information and the location information comprises:
periodically measuring and transmitting, by a patch-type thermometer attached to the user's body, the body temperature of the user; and
receiving, by a beacon gateway which is the electronic device installed in a predetermined place, the body temperature information transmitted by the patch-type thermometer and transmitting the installation location information of the beacon gateway and the body temperature information to the data server.
